(19) United States
(12) Patent Application Publication
(10) Pub. No.: US 2005/0106589 A1
(43) Pub. Date: May 19, 2005

(54) Compositions and methods for releasing nucleic acids from solid phase binding materials

(76) Inventors: Hashem Akhavan-Tafti, Howell, MI (US); Renuka de Silva, Northville, MI (US); Sarada Siripurapu, Novi, MI (US); Richard Handley, Canton, MI (US)

Correspondence Address: LUMIGEN, INC., 22900 W. EIGHT MILE ROAD, SOUTHFIELD, MI, 48034, US

(21) Appl. No.: 10/891,880

(22) Filed: Jul. 15, 2004

Related U.S. Application Data
(63) Continuation-in-part of application No. 10/714,763, filed on Nov. 17, 2003.
(63) Continuation-in-part of application No. 10/715,284, filed on Nov. 17, 2003.

(51) Int. Cl. C12Q 1/68; C07H 21/04

(52) U.S. Cl. 435/6; 536/25.4

(57) ABSTRACT

Methods of isolating nucleic acids are disclosed comprising binding the nucleic acid to solid phase binding materials and releasing the bound nucleic acid from the solid phase by elution with a novel reagent composition. Compositions feature a high ionic strength buffer or an added hydrophilic organic co-solvent or both. Preferred solid phase materials for use with the methods and compositions of the invention comprise a quaternary onium nucleic acid binding portion.

### CROSS REFERENCE TO RELATED APPLICATION

The present application is a continuation-in-part of Applicants' co-pending U.S. application Ser. No. 10/714,763, filed on Nov. 17, 2003 and U.S. application Ser. No. 10/715,284, filed on Nov. 17, 2003.

### FIELD OF THE INVENTION

The present invention relates to the use of novel compositions for releasing nucleic acids bound to solid phase materials used to bind, isolate, or purify nucleic acids.

### BACKGROUND OF THE INVENTION

Molecular diagnostics and modern techniques in molecular biology (including reverse transcription, cloning, restriction analysis, amplification, and sequence analysis), require that nucleic acids used in these techniques be substantially free of contaminants and interfering substances. Undesirable contaminants include macromolecular substances such as enzymes, other types of proteins, polysaccharides, polynucleotides, oligonucleotides, nucleotides, lipids, low molecular weight enzyme inhibitors, or non-target nucleic acids, enzyme cofactors, salts, chaotropes, dyes, metal salts, buffer salts and organic solvents.

Obtaining target nucleic acid substantially free of contaminants for molecular biological applications is difficult due to the complex sample matrix in which target nucleic acids are found. Such samples include, e.g., cells from tissues, cells from bodily fluids, blood, bacterial cells in culture, agarose gels, polyacrylamide gels, or solutions resulting from amplification of target nucleic acids. Sample matrices often contain significant amounts of contaminants which must be removed from the nucleic acid(s) of interest before the nucleic acids can be used in molecular biological or diagnostic techniques.

Conventional techniques for isolating target nucleic acids from mixtures produced from cells and tissues as described above, require the use of hazardous chemicals such as phenol, chloroform, and ethidium bromide. Phenol/chloroform extraction is used in such procedures to extract contaminants from mixtures of target nucleic acids and various contaminants. Alternatively, cesium chloride-ethidium bromide gradients are used according to methods well known in the art. See, e.g., Molecular Cloning, ed. by Sambrook et al. (1989), Cold Spring Harbor Press, pp. 1.42-1.50. The latter methods are generally followed by precipitation of the nucleic acid material remaining in the extracted aqueous phase by adding ethanol or 2-propanol to the aqueous phase to precipitate nucleic acid. The precipitate is typically removed from the solution by centrifugation, and the resulting pellet of precipitate is allowed to dry before being resuspended in water or a buffer solution for further use.

Simpler and faster methods have been developed which use various types of solid phases to separate nucleic acids from cell lysates or other mixtures of nucleic acids and contaminants. Such solid phases include chromatographic resins, polymers and silica or glass-based materials in various shapes and forms such as fibers, filters and coated containers. When in the form of small particulates, magnetic cores are sometimes provided to assist in effecting separation.

One type of solid phase used in isolating nucleic acids comprises porous silica gel particles designed for use in high performance liquid chromatography (HPLC). The surface of the porous silica gel particles is functionalized with anion-exchangers to exchange with plasmid DNA under certain salt and pH conditions. See, e.g. U.S. Pat. Nos. 4,699,717, and 5,057,426. Plasmid DNA bound to these solid phase materials is eluted in an aqueous solution containing a high concentration of a salt. The nucleic acid solution eluted therefrom must be treated further to remove the salt before it can be used in downstream processes.

Other silica-based solid phase materials comprise controlled pore glass (CPG), filters embedded with silica particles, silica gel particles, diatomaceous earth, glass fibers or mixtures of the above. Each silica-based solid phase material reversibly binds nucleic acids in a sample containing nucleic acids in the presence of chaotropic agents such as sodium iodide (NaI), guanidinium thiocyanate or guanidinium chloride. Such solid phases bind and retain the nucleic acid material while the solid phase is subjected to centrifugation or vacuum filtration to separate the matrix and nucleic acid material bound thereto from the remaining sample components. The nucleic acid material is then freed from the solid phase by eluting with water or a low salt elution buffer. Commercially available silica-based solid phase materials for nucleic acid isolation include, e.g., Wizard™ DNA purification systems products (Promega, Madison, Wis.), the QiaPrep™ DNA isolation systems (Qiagen, Santa Clarita, Calif.), High Pure (Roche), and GFX Micro Plasmid Kit, (Amersham).

Polymeric resins in the form of particles are also in widespread use for isolation and purification of nucleic acids. Carboxylate-modified polymeric particles (Bangs, Agencourt) are known. Polymers having quaternary ammonium head groups are disclosed in European Patent Application Publ. No. EP 1243649A1. The polymers are inert carrier particles having covalently attached linear non-crosslinked polymers. This type of polymeric solid phase is commonly referred to as a tentacle resin. The linear polymers incorporate quaternary tetraalkylammonium groups. The alkyl groups are specified as methyl or ethyl groups (Column 4, lines 52-55). Longer alkyl groups are deemed undesirable.

Other solid phase materials for binding nucleic acids based on the anion exchange principle are in present use. These include a silica based material having DEAE head groups (Qiagen) and a silica-NucleoBond AX (Becton Dickinson, Roche-Genopure) based on the chromatographic support described in EP0496822B1. Polymer resins with polymeric-trialkylammonium groups are disclosed in EP 1243649 (GeneScan). Carboxyl-modified polymers for DNA isolation are available from numerous suppliers. Nucleic acids are attracted under high salt conditions and released under low ionic strength conditions. A polymeric microcarrier bead having a cationic trimethylamine exterior is described in U.S. Pat. No. 6,214,618. The beads have a relatively large diameter and are useful as a support for cell attachment and growth in culture.

Polymeric beads having a tributylphosphonium head group have been described for use as phase transfer catalysts in a three phase system. The beads were prepared from a crosslinked polystyrene. (J. Chem. Soc. Perkin Trans. II, 1827-1830, (1983)). Polymer beads having a pendant trialkylphosphonium group linked to a cross-linked polystyrene resin through alkylene chains and alkylene ether chains have also been described (Tomoi, et al., Makromolekulare Chemie, 187(2), 357-65 (1986); Tomoi, et al., Reactive Polymers, Ion Exchangers, Sorbents, 3(4), 341-9 (1985)). Mixed quaternary ammonium/phosphonium insoluble polymers based on cross-linked polystyrene resins are disclosed as catalysts and biocides (Davidescu, et al., Chem. Bull. Techn. Univ. Timisoara, 40(54), 63-72 (1995); Parvulescu, et al., Reactive & Functional Polymers, 33(2,3), 329-36 (1997).

Magnetically responsive particles have also been developed for use as solid phases in isolating nucleic acids. Several different types of magnetically responsive particles designed for isolation of nucleic acids are known in the art and commercially available from several sources. Magnetic particles which reversibly bind nucleic acid materials directly include MagneSil™ particles (Promega). Magnetic particles are also known that reversibly bind mRNA via covalently attached avidin or streptavidin having an attached oligo dT tail for hybridization with the poly A tail of mRNA.

Various types of magnetically responsive silica-based particles are known for use as solid phases in nucleic acid binding isolation methods. One such particle type is a magnetically responsive glass bead, preferably of a controlled pore size available as Magnetic Porous Glass (MPG) particles from CPG, Inc. (Lincoln Park, N.J.); or porous magnetic glass particles described in U.S. Pat. No. 4,395,271; 4,233,169; or 4,297,337. Another type of magnetic particle useful for binding and isolation of nucleic acids is produced by incorporating magnetic materials into the matrix of polymeric silicon dioxide compounds. (German Patent DE4307262A1) Magnetic particles comprising iron oxide nanoparticles embedded in a cellulose matrix having quaternary ammonium group is produced commercially by Cortex Biochem (San Leandro, Calif.) as MagaCell-Q™.

Particles or beads having inducible magnetic properties comprise small particles of transition metals such as iron, nickel, copper, cobalt and manganese to form metal oxides which can be caused to have transitory magnetic properties in the presence of magnet. These particles are termed paramagnetic or superparamagnetic. To form paramagnetic or superparamagnetic beads, metal oxides have been coated with polymers which are relatively stable in water. U.S. Pat. No. 4,554,088 discloses paramagnetic particles comprising a metal oxide core surrounded by a coat of polymeric silane. U.S. Pat. No. 5,356,713 discloses a magnetizable microsphere comprised of a core of magnetizable particles surrounded by a shell of a hydrophobic vinylaromatic monomer. U.S. Pat. No. 5,395,688 discloses a polymer core which has been coated with a mixed paramagnetic metal oxide-polymer layer. Another method utilizes a polymer core to adsorb metal oxide such as for example in U.S. Pat. No. 4,774,265. Magnetic particles comprising a polymeric core particle coated with a paramagnetic metal oxide particle layer is disclosed in U.S. Pat. No. 5,091,206. The particle is then further coated with additional polymeric layers to shield the metal oxide layer and to provide a reactive coating. U.S. Pat. No. 5,866,099 discloses the preparation of magnetic particles by co-precipitation of mixtures of two metal salts in the presence of a protein to coordinate the metal salt and entrap the mixed metal oxide particle. Numerous exemplary pairs of metal salts are described. U.S. Pat. No. 5,411,730 describes a similar process where the precipitated mixed metal oxide particle is entrapped in dextran, an oligosaccharide.

Alumina (aluminum oxide) particles for irreversible capture of DNA and RNA is disclosed in U.S. Pat. No. 6,291,166. Bound nucleic acid is available for use in solid phase amplification methods such as PCR.

DNA bound to these solid phase materials is eluted in an aqueous solution containing a high concentration of a salt. The nucleic acid solution eluted therefrom must be treated further to remove the salt before it can be used in downstream processes. Nucleic acids bound to silica-based material, in contrast, are freed from the solid phase by eluting with water or a low salt elution buffer. U.S. Pat. No. 5,792,651 describes a composition for chromatographic isolation of nucleic acids which enhances the ability of the nucleic acid in transfection in cells. The composition comprises an aqueous solution containing 2-propanol and optional salts and buffer materials.

Yet other magnetic solid phase materials comprising agarose or cellulose particles containing magnetic microparticle cores are reported to bind and retain nucleic acids upon treatment with compositions containing high concentrations of salts and polyalkylene glycol (e.g. U.S. Pat. No. 5,898,071 and PCT Publication WO02066993). Nucleic acid is subsequently released by treatment with water or low ionic strength buffer.

### SUMMARY OF THE INVENTION

It is an object of the present invention to provide methods for isolating nucleic acids using solid phase nucleic acid binding materials and reagent compositions of the present invention. It is another object of the present invention to provide methods for binding and releasing the nucleic acids from solid phase materials with reagent compositions of the present invention.

It is another object of the present invention to provide methods for isolating nucleic acids using solid phase nucleic acid binding materials by releasing bound nucleic acid with reagent compositions of the present invention containing alkaline amine buffers and hydrophilic organic solvents and optionally containing salts.

In another object of the present invention there are provided reagent compositions for releasing bound nucleic acids from solid phase materials. The compositions of the invention function to release or elute bound nucleic acids both from the present cleavable solid phase materials and from other conventional solid phase materials, including those with cationic, anionic or neutral surfaces.

### DETAILED DESCRIPTION OF THE INVENTION

Definitions

Alkyl—A branched, straight chain or cyclic hydrocarbon group containing from 1-20 carbons which can be substituted with 1 or more substituents other than H. Lower alkyl as used herein refers to those alkyl groups containing up to 8 carbons.

Aralkyl—An alkyl group substituted with an aryl group.

Aryl—An aromatic ring-containing group containing 1 to 5 carbocyclic aromatic rings, which can be substituted with 1 or more substituents other than H.

Magnetic particle—a particle, microparticle or bead that is responsive to an external magnetic field. The particle may itself be magnetic, paramagnetic or superparamagnetic. It may be attracted to an external magnet or applied magnetic field as when using ferromagnetic materials. Particles can have a solid core portion that is magnetically responsive and is surrounded by one or more non-magnetically responsive layers. Alternately the magnetically responsive portion can be a layer around or can be particles disposed within a non-magnetically responsive core.

Oligomer, oligonucleotide—as used herein will refer to a compound containing a phosphodiester internucleotide linkage and a 5′-terminal monophosphate group. The nucleotides can be the normally occurring ribonucleotides A, C, G, and U or deoxyribonucleotides, dA, dC, dG and dT.

Polynucleotide—A polynucleotide can be DNA, RNA or a synthetic DNA analog such as a PNA. Double-stranded hybrids of any of these three types of chains are also within the scope of the term.

Primer—refers to an oligonucleotide used to direct the site of ligation and is required to initiate the ligation process. Primers are of a length sufficient to hybridize stably to the template and represent a unique sequence in the template. Primers will usually be about 15-30 bases in length. Labeled primers containing detectable labels or labels which allow solid phase capture are within the scope of the term as used herein.

Release, elute—to remove a substantial portion of a material bound to the surface or pores of a solid phase material by contact with a solution or composition.

Sample—A fluid containing or suspected of containing nucleic acids. Typical samples which can be used in the methods of the invention include bodily fluids such as blood, plasma, serum, urine, semen, saliva, cell lysates, tissue extracts and the like. Other types of samples include solvents, seawater, industrial water samples, food samples and environmental samples such as soil or water, plant materials, cells originated from prokaryotes, eukaryotes, bacteria, plasmids and viruses.

Solid phase material—a material having a surface to which can attract nucleic acid molecules. Materials can be in the form of microparticles, fibers, beads, membranes, and other supports such as test tubes and microwells.

Substituted—Refers to the replacement of at least one hydrogen atom on a group by a non-hydrogen group. It should be noted that in references to substituted groups it is intended that multiple points of substitution can be present unless clearly indicated otherwise.

Template, test polynucleotide, and target are used interchangeably and refer to the nucleic acid whose length is to be replicated.

Nucleic acids are extracted isolated and otherwise purified from various sample types by a variety of techniques. Many of these techniques rely on selective adsorption onto a surface of a material with some affinity for nucleic acids. After washing steps to remove other, less strongly bound components, the solid phase is treated with a solution to remove or elute bound nucleic acid(s). Applicants have developed novel reagent compositions useful for eluting nucleic acids that have been bound onto solid phase binding materials. The solid phase binding materials with which the present compositions are useful include conventional silica based materials, functionalized silica bearing covalently attached surface functional groups such as carboxy groups, amino groups and hydroxy groups, carbohydrate based materials, and polymeric materials as well as the quaternary and ternary onium salt type materials described below and in Applicants' co-pending U.S. application Ser. Nos. 10/714,763 and 10/715,284, the disclosures of which are incorporated herein by reference.

Solid phase materials for binding nucleic acids for use with the compositions and methods of the present invention can be in the form of particles, microparticles, fibers, beads, membranes, and other supports such as test tubes and microwells. The materials further comprise an nucleic acid binding surface which permits capture and binding of nucleic acid molecules of varying lengths. By surface is meant not only the external periphery of the solid phase material but also the surface of any accessible porous regions within the solid phase material.

The present compositions encompass a family of aqueous buffer solutions of neutral to alkaline pH. One group comprises an aqueous solution of an amine buffer having a pH of 7-9 wherein the concentration of the amine is at least 0.1 M and preferably at least 0.4 M and more preferably at least 1 M. Buffer solutions of this type contain no other added salts such as NaCl or KCl, relying on the buffer components to achieve the elution efficiency. Amines useful as buffering components include aliphatic amines, aliphatic amino alcohols and sulfonated aliphatic amines. Exemplary amines include diethylamine, triethylamine, imidazole, amino acids (e.g., glycine, glycylglycine, N-(Carbamoylmethyl)iminodiacetic acid (ADA),).

Exemplary amino alcohol compounds include tris(hydroxymethyl)aminomethane (TRIS), tris(hydroxymethyl)methylaminopropane (Bis-TRIS), 2-methyl-2-amino-1-propanol (AMP), 2-amino-2-methyl-1,3-propanediol (AMPD), ethanolamine, diethanolamine, and triethanolamine.

Exemplary sulfonated aliphatic amines include 3-N-morpholinopropanesulfonic acid (MOPS), 3-N-(trishydroxymethyl)methylaminopropanesulfonic acid (TAPS), 3-N-(trishydroxymethyl)methylamino-2-hydroxypropanesulfonic acid (TAPSO), N-2-hydroxyethylpiperazine-N′-2-ethanesulfonic acid (HEPES), 1,4-piperazinebis(ethanesulfonic acid) (PIPES), 4-morpholinoethanesulfonic acid (MES), 2-(tris(hydroxymethyl)methylamino)ethanesulfonic acid (TES), N,N-bis(2-hydroxyethyl)-2-aminoethane-sulfonic acid (BES), N-cyclohexyl-2-aminoethane-sulfonic acid (CHES), 2-(Carbamoylmethylamino)-ethanesulfonic acid (ACES), N,N-bis(2-hydroxy-ethyl)glycine (bicine), 3-(Cyclohexylamino)-1-propanesulfonic acid (CAPS), N-(2-Hydroxy-ethyl)piperazine-N′-(2-hydroxypropanesulfonic acid) (HEPPSO), piperazine-N,N′-bis(2-hydroxypropane-sulfonic acid) (POPSO), and N-tris(hydroxymethyl)-methylglycine (tricine).

In a preferred composition, the buffer also contains 0.1-50% of a hydrophilic organic co-solvent, more preferably from 1-20% of the solvent. Reference to hydrophilic organic solvent is meant to include organic compounds having solubility in water or aqueous solutions of at least 0.1%, preferably at least 1% and more preferably at least 10%. Exemplary hydrophilic organic co-solvents include C₁-C₄ alcohols, ethylene glycol, propylene glycol, glycerol, water soluble mercaptans, 2-mercaptoethanol, dithiothreitol, furfuryl alcohol, 2,2,2-trifluoroethanol, acetone, THF, and p-dioxane. A preferred embodiment uses a composition containing 2-mercaptoethanol or dithiothreitol as the hydrophilic organic co-solvent.

Another group of compositions comprises an aqueous solution of an amine buffer having a pH of 7-9 wherein the concentration of the amine is at least 0.01 M and at least one monovalent or divalent halide salt or acetate salt at a concentration of 0.1-3 M. Representative salts include halides and acetate salts of NH₄, and metals Li, Na, K, Rb, Cs, Ca, Mg, and Zn. A preferred halide is chloride. The combined concentration of buffer and salt is at least 0.1 M. An exemplary buffer of this type, sold as a PCR buffer 20× concentrate, contains 0.4 M tris-HCl, pH 8.4, 1 M KCl and 0.05 M MgCl₂. Members of this group of compositions can optionally further comprise a hydrophilic organic co-solvent at 0.01-50%. Exemplary hydrophilic organic co-solvents include C₁-C₄ alcohols, ethylene glycol, propylene glycol, glycerol, water soluble mercaptans, 2-mercaptoethanol, dithiothreitol, furfuryl alcohol, 2,2,2-trifluoroethanol, acetone, THF, and p-dioxane. A preferred embodiment uses a composition containing 2-mercaptoethanol or dithiothreitol as the hydrophilic organic co-solvent. In another preferred composition the amount of the hydrophilic organic co-solvent is from 0.1-50% of the composition. More preferably the amount of the hydrophilic organic co-solvent is from 1-20% of the solvent.

A benefit of the novel compositions is the ability to use solutions of the eluted nucleic acid directly in many downstream molecular biology processes without having to first precipitate and collect the nucleic acid. Methods of using the compositions to elute or release bound nucleic acids as part of a process of isolating or purifying a nucleic acid from a sample also form another part of the invention and are disclosed in more detail below.

All of the disclosed compositions have been found to be effective in removing bound nucleic acid from solid phase materials having quaternary onium groups for binding nucleic acid. The use of any of these compositions in a method of isolating nucleic acids using such solid phase materials constitutes one aspect of the present invention.

It has also been found that nucleic acid bound to other known nucleic acid binding supports can be released from these solid supports by contacting them with novel reagent compositions of the present invention comprising a buffer solution having a pH of about 7-9 wherein the buffering component is present in a concentration of at least 0.1 M and preferably at least 0.4 M, and optionally comprising a hydrophilic organic co-solvent at 0.1-50%.

In one aspect of the invention there is provided a method of isolating a nucleic acid from a sample comprising:
a) providing a solid phase comprising:
a matrix selected from silica, glass, insoluble synthetic polymers, and insoluble polysaccharides, and
b) combining the solid phase with the sample containing the nucleic acid to bind the nucleic acid to the solid phase;
c) separating the sample from the solid phase; and
d) releasing the nucleic acid from the solid phase by contacting the solid phase with a reagent composition comprising an aqueous buffer solution having a pH of 7-9, wherein the concentration of the buffer is at least 0.1 M, and a hydrophilic organic co-solvent at 0.1-50%.

Among the conventional solid phase materials usable in conjunction with the present elution compositions are silica particles, silica-coated surfaces including membranes, silica having surface functionalization such as amine-functionalized and carboxy-functionalized silica, synthetic polymer beads and particles known in the art of nucleic acid purification, agarose or cellulose particles, and agarose or cellulose-coated silica particles. Magnetic particles coated with any of the foregoing materials function similarly and are also usable in the conjunction with the present compositions and methods.

The compositions of the present invention find particular utility in combination with solid phase binding materials having a quaternary onium group of the formula QR₂⁺X⁻ or QR₃⁺X⁻ attached on a surface of the matrix wherein the quaternary onium group is selected from ternary sulfonium groups, quaternary ammonium, and phosphonium groups wherein R is selected from C₁-C₂₀ alkyl, aralkyl and aryl groups, and X is an anion. Preferably the onium group is selected from the quaternary phosphonium groups ⁺ PR₃X⁻ wherein R is as defined above.

In another aspect of the invention there is provided a method of isolating a nucleic acid from a sample comprising:
a) providing a solid phase comprising:
   a matrix selected from silica, glass, insoluble synthetic polymers, and insoluble polysaccharides, and an onium group attached on a surface of the matrix selected from a ternary sulfonium group of the formula QR₂⁺X⁻ where R is selected from C₁-C₂₀ alkyl, aralkyl and aryl groups, a quaternary ammonium group of the formula NR₃⁺X⁻ wherein the quaternary onium group wherein R is selected from C₁-C₂₀ alkyl, aralkyl and aryl groups, and a quaternary phosphonium group PR₃⁺X⁻ wherein R is selected from C₁-C₂₀ alkyl, aralkyl and aryl groups, and wherein X is an anion,
b) combining the solid phase with the sample containing the nucleic acid to bind the nucleic acid to the solid phase;
c) separating the sample from the solid phase; and
d) releasing the nucleic acid from the solid phase by contacting the solid phase with a reagent composition comprising an aqueous solution having a pH of 7-9, 0.1-3 M metal halide salt or acetate salt and a hydrophilic organic co-solvent at 0.1-50%.

Representative salts include halides and acetate salts of NH₄, and metals Li, Na, K, Rb, Cs, Ca, Mg, and Zn. A preferred halide is chloride.

Exemplary hydrophilic organic co-solvents include C₁-C₄ alcohols, ethylene glycol, propylene glycol, glycerol, water soluble mercaptans, 2-mercaptoethanol, dithiothreitol, furfuryl alcohol, 2,2,2-trifluoroethanol, acetone, THF, and p-dioxane. In a preferred method the onium group on the solid phase is selected from the quaternary phosphonium groups ⁺PR₃X⁻ wherein R is as defined above.

As disclosed in the aforementioned co-pending U.S. application Ser. No. 10/714, 763 and 10/715,284, Applicants have developed solid phase materials which bind nucleic acids and have a cleavable linker portion which can be cleaved to release the bound nucleic acids. These cleavable solid phase materials also permit elution of nucleic bound thereto through contact with compositions of the present invention without cleaving the linker group. The materials can be in the form of microparticles, fibers, beads, membranes, and other supports such as test tubes and microwells that have sufficient surface area to permit efficient binding. Solid phase materials useful in the methods of the present invention in the form of microparticles can further comprise a magnetic core portion. Generally, particles and magnetically responsive microparticles are preferred in the present invention.

All solid phase nucleic acid binding materials useful in the methods of the present invention comprise a matrix which defines its size, shape, porosity, and mechanical properties, and covalently linked nucleic acid binding groups. The three most common kinds of matrix are silica or glass, insoluble synthetic polymers, and insoluble polysaccharides. The solid phase can further comprise a magnetically responsive portion.

Polymers are homopolymers or copolymers of one or more ethylenically unsaturated monomer units and can be crosslinked or non-crosslinked. Preferred polymers are polyolefins including polystyrene and the polyacrylic-type polymers. The latter comprise polymers of various substituted acrylic acids, amides and esters, wherein the acrylic monomer may or may not have alkyl substituents on the 2- or 3-carbon.

The nucleic acid binding groups contained in the cleavable and noncleavable solid phase binding materials useful in the methods of the present invention attract and bind nucleic acids, polynucleotides and oligo-nucleotides of various lengths and base compositions or sequences. Nucleic acid binding groups include carboxyl, amine and ternary or quaternary onium groups. Amine groups can be NH₂, alkylamine, and dialkylamine groups. Ternary or quaternary onium groups include quaternary trialkylammonium groups (-QR₃⁺), phosphonium groups (-QR₃⁺) including trialkylphosphonium or triarylphosphonium or mixed alkyl aryl phosphonium groups, and ternary sulfonium groups (-QR₂⁺). The solid phase can contain more than one kind of nucleic acid binding group as described herein. Solid phase materials containing ternary or quaternary onium groups -QR₂⁺ or -QR₃⁺ wherein the R groups are alkyl of at least four carbons are especially effective in binding nucleic acids, but alkyl groups of as little as one carbon are also useful as are aryl groups. Such solid phase materials retain the bound nucleic acid with great tenacity and resist removal or elution of the nucleic acid under most conditions used for elution known in the prior art. Most known elution conditions of both low and high ionic strength are ineffective in removing bound nucleic acids. Unlike conventional anion-exchange resins containing DEAE and PEI groups, the ternary or quaternary onium solid phase materials remain positively charged regardless of the pH of the reaction medium.

Cleavable solid phase materials comprise a solid support portion comprising a matrix selected from silica, glass, insoluble synthetic polymers, and insoluble polysaccharides to which is attached on a surface a nucleic acid binding portion for attracting and binding nucleic acids, the nucleic acid binding portion (NAB) being linked by a cleavable linker portion to the solid support portion.

[image]

In one embodiment the NAB is a ternary onium group of the formula QR₂⁺X⁻ wherein Q is a S atom or a quaternary onium group QR₃⁺X⁻ wherein Q is a N or P atom, R is selected from alkyl, aralkyl and aryl groups and X is an anion. When Q is a nitrogen atom, the R groups will each contain from 4-20 carbon atoms. When Q is a sulfur or phosphorus atom, the R groups can have from 1-20 carbon atoms.

[image]

A preferred cleavable solid phase is derived from commercially available polystyrene type polymers such as those of the kind referred to as Merrifield resin (crosslinked). In these polymers a percentage of the styrene units contain a reactive group, typically a chloromethyl or hydroxymethyl group as a means of covalent attachment. Replacement of some of the chlorines by reaction with a sulfide (R₂S) or a tertiary amine or phosphine produces the solid phase materials of the invention. A polymer prepared in accordance with this definition can be depicted by the formula (1) below when all of the reactive chloromethyl groups have been converted to ternary or quaternary onium groups. It is not necessary for all such groups to be converted so that polymeric solid phases of the invention will often contain a mixture of the onium group and the chloromethyl group. 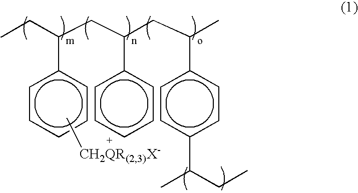
In the formula above, m, n, and o denote the mole percentage of each monomeric unit in the polymer and can take the values m from 0.1% to 100%, n from 0 to 99%, and o from 0 to 10%. More preferably m is from 1% to 20%, n is from 80 to 99%, and o is from 0 to 10%.

In another embodiment, a cleavable solid phase is derived from a commercially available crosslinked Merrifield resin having a percentage of the styrene units contain a reactive chloroacetyl or chloropropionyl group for covalent attachment. Ternary or quaternary onium polymers of the invention prepared from these starting polymers have the formula: 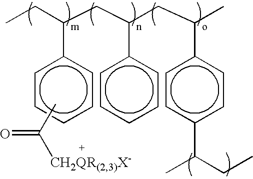
where Q, R, X, m, n, and o are as defined above.

Numerous other art-known polymeric resins can be used as the solid matrix in preparing cleavable solid phase materials. Polymeric resins are available from commercial suppliers such as Advanced ChemTech (Louisville, Ky.) and NovaBiochem. The resins are generally based on a crosslinked polymeric particle having a reactive functional group. Many suitable polymeric resins used in solid supported peptide synthesis as described in the Advanced ChemTech 2002 Catalog, pp. 105-140 are appropriate starting materials. Polymers having reactive NH₂, NH—NH₂, OH, SH, CHO, COOH, CO₂CH═CH₂, NCO, Cl, Br, SO₂CH═CH₂, SO₂Cl, SO₂NH₂, acylimidazole, oxime (C═N—OH), succinimide ester groups are each commercially available for use in preparation of polymeric solid phases of the invention. As is shown below in numerous examples it is sometimes necessary or desirable to provide a means of covalently joining a precursor polymer resin to the ternary or quaternary onium group. This will generally comprise a chain or ring group of 1-20 atoms selected from alkylene, arylene or aralkylene groups. The chain or ring can also contain O, S, or N atoms and carbonyl groups in the form of ketones, esters, thioesters, amides, urethanes, carbonates, xanthates, ureas, imines, oximes, sulfoxides and thioketones.

As used herein, magnetic microparticles are particles that can be attracted and manipulated by a magnetic field. The magnetic microparticles used in the method of the present invention comprise a magnetic metal oxide core, which is generally surrounded by an adsorptively or covalently bound layer to which a nucleic acid binding layer is covalently bound through selected coupling chemistries, thereby coating the surface of the microparticles with ternary sulfonium, quaternary ammonium, or quaternary phosphonium functional groups. The magnetic metal oxide core is preferably iron oxide, wherein iron is a mixture of Fe²⁺ and Fe³⁺. Magnetic microparticles comprising an iron oxide core, as described above, without a silane coat can also be used in the method of the present invention. Magnetic particles can also be formed as described in U.S. Pat. No. 4,654,267 by precipitating metal particles in the presence of a porous polymer to entrap the magnetically responsive metal particles. Magnetic metal oxides preparable thereby include Fe₃O₄, MnFe₂O₄, NiFe₂O₄, and CoFe₂O₄. Other magnetic particles can also be formed as described in U.S. Pat. No. 5,411,730 by precipitating metal oxide particles in the presence of a the oligosaccharide dextran to entrap the magnetically responsive metal particles. Yet another kind of magnetic particle is disclosed in the aforementioned U.S. Pat. No. 5,091,206. The particle comprises a polymeric core particle coated with a paramagnetic metal oxide particle layer and additional polymeric layers to shield the metal oxide layer and to provide a reactive coating. Preparation of magnetite containing chloromethylated Merrifield resin is described in a publication (Tetrahedron Lett., 40 (1999), 8137-8140).

Commercially available magnetic silica or magnetic polymeric particles can be used as the starting materials in preparing cleavable magnetic particles in accordance with the present invention. Suitable types of polymeric particles having surface carboxyl groups are known by the tradenames SeraMag™ (Seradyn) and BioMag™ (Polysciences and Bangs Laboratories). A suitable type of silica magnetic particles is known by the tradename MagneSil™ (Promega). Silica magnetic particles having carboxy or amino groups at the surface are available from Chemicell GmbH (Berlin).

The cleavable linker portion is preferably an organic group selected from straight chains, branched chains and rings and comprises from 1 to 100 atoms and more preferably from 1 to about 50 atoms. The atoms are preferably selected from C, H, B, N, O, S, Si, P, halogens and alkali metals. An exemplary linker group is a hydrolytically cleavable group which is cleaved by hydrolysis. Carboxylic esters and anhydrides, thioesters, carbonate esters, thiocarbonate esters, urethanes, imides, sulfonamides, and sulfonimides are representative as are sulfonate esters. Another exemplary class of linker groups are those groups which undergo reductive cleavage. One representative group is an organic group containing a disulfide (S—S) bond which is cleaved by thiols such as ethanethiol, mercaptoethanol, and DTT. Another representative group is an organic group containing a peroxide (O—O) bond. Peroxide bonds can be cleaved by thiols, amines and phosphines. 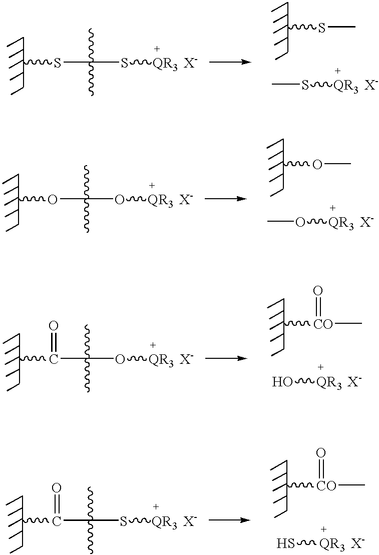
While many of the particular structure drawings represent only a quaternary onium group for convenience it should be understood that the analogous ternary sulfonium group is also meant to be represented as well.

Exemplary photochemically cleavable linker groups include nitro-substituted aromatic ethers and esters of the formula 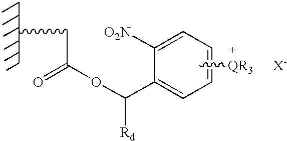
where R_{d} is H, alkyl or phenyl, and more particularly 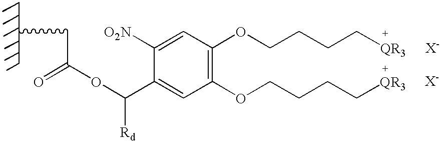
Ortho-nitrobenzyl esters are cleaved by ultraviolet light according to the well known reaction

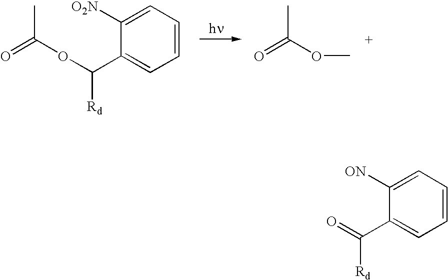

Exemplary enzymatically cleavable linker groups include esters which are cleaved by esterases and hydrolases, amides and peptides which are cleaved by proteases and peptidases, glycoside groups which are cleaved by glycosidases.

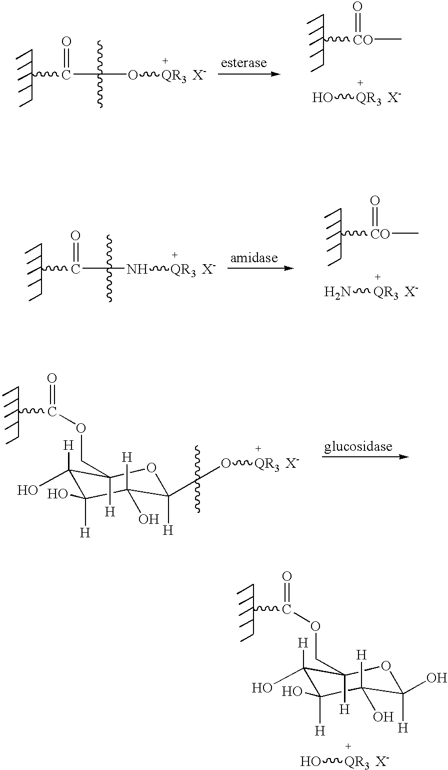

Solid phase materials having a linker group comprising a cleavable 1,2-dioxetane moiety are also within the scope of the inventive nucleic acid binding materials. Such materials contain a dioxetane moiety which can be triggered to fragment by a chemical or enzymatic agent. Removal of a protecting group to generate an oxyanion promotes decomposition of the dioxetane ring. Fragmentation occurs by cleavage of the peroxidic O—O bond as well as the C—C bond according to a well known process. 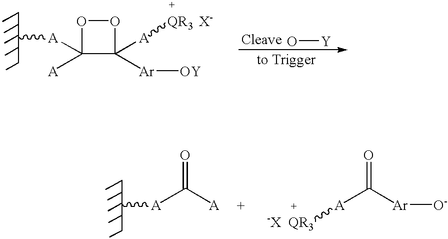
In the alternative, the linked onium group can be attached to the aryl group Ar or to the cleavable group Y. In a further alternative, the linkages to the solid phase and ternary or quaternary onium groups are reversed from the orientation shown.

In the foregoing exemplary reactions, the groups A represent stabilizing substituents selected from alkyl, cycloalkyl, polycycloalkyl, polycycloalkenyl, aryl, aryloxy and alkoxy groups. Ar represents an aryl ring group. Preferred aryl ring groups are phenyl and naphthyl groups. The aryl ring can contain additional substituents, in particular halogens, alkoxy and amine groups. The Y group is a group or atom which is removable by a chemical agent or enzyme. Suitable OY groups include OH, OSiR³₃, wherein R³ is selected from alkyl and aryl groups, carboxyl groups, phosphate salts, sulfate salts, and glycoside groups. Numerous triggerable dioxetane structures are well known in the art and have been the subject of a large number of patents. An exemplary cleavable dioxetane linker and its cleavage is depicted below.

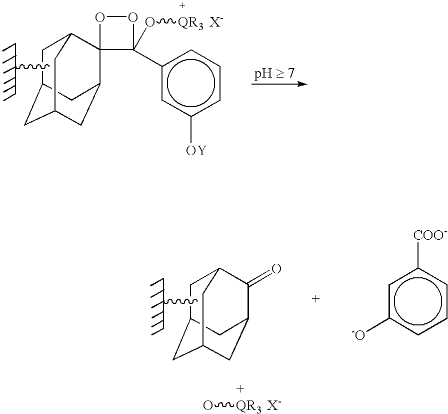

Removal of the protecting group Y triggers a fragmentation of the dioxetane ring and thereby separates the solid matrix and onium groups. Under alkaline reaction conditions the resulting aryl ester undergoes further hydrolysis.

Solid phase materials having a linker group comprising an electron-rich C—C double bond which can be converted to an unstable 1,2-dioxetane moiety are another group of cleavable nucleic acid binding materials. At least one of the substituents (A′) on the double bond is attached to the double bond by means of an O, S, or N atom. Reaction of electron-rich double bonds with singlet oxygen produces an unstable 1,2-dioxetane ring group which spontaneously fragments at ambient temperatures to generate two carbonyl fragments.

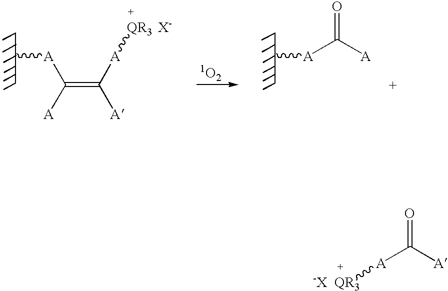

Another group of solid phase materials having a cleavable linker group have as the cleavable moiety a ketene dithioacetal as disclosed in PCT Publication WO 03/053934. Ketene dithioacetals undergo oxidative cleavage by enzymatic oxidation with a peroxidase enzyme and hydrogen peroxide. 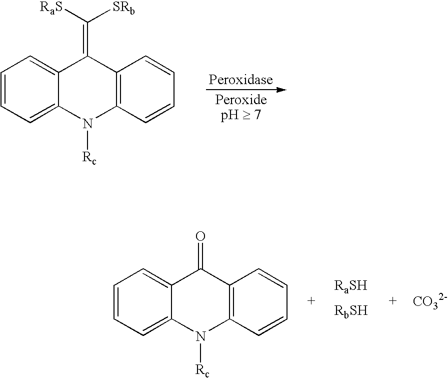
The cleavable moiety has the structure shown, including analogs having substitution on the acridan ring, wherein Rₐ R_{b} and R_{c} are each organic groups containing from 1 to about 50 non-hydrogen atoms selected from C, N, O, S, P, Si and halogen atoms and wherein Rₐ and R_{b} can be joined together to form a ring.

Solid phase materials having a ketene dithioacetal cleavable linker group can have any of the formulas: 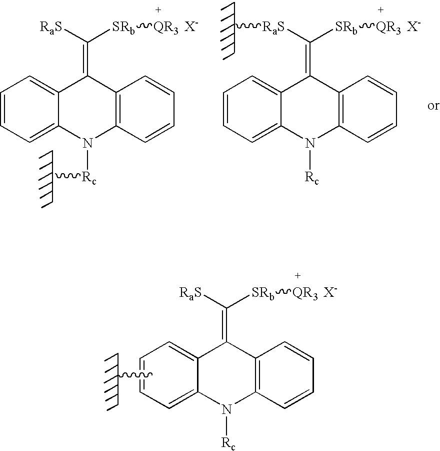
as well as the analogous structures where the order of attachment of the solid matrix and onium groups to the cleavable linker moiety is reversed from those shown.

Another group of solid phase materials having a cleavable linker group have as the cleavable moiety an alkylene group of at least one carbon atom bonded to a trialkyl or triarylphosphonium group. 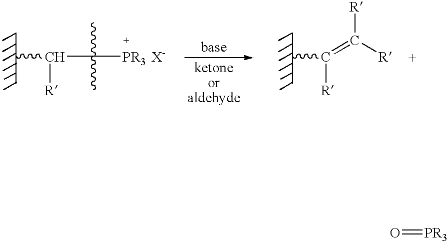
Materials of this group are cleavable by means of a Wittig reaction with a ketone or aldehyde. Reaction of a quaternary phosphonium compound with a strong base in an organic solvent deprotonates the carbon atom bonded to the phosphorus creating a phosphorus ylide. Reaction of the ylide with a carbonyl containing compound such as a ketone or aldehyde forms a double bond and the phosphine oxide. The link between the phosphonium group and the solid phase is broken in the process.

A further aspect of the invention comprises methods of isolating and purifying nucleic acids using the cleavable solid phase binding materials. In one embodiment there is provided a method of isolating a nucleic acid from a sample comprising:
a) providing a solid phase comprising:
   a solid support portion comprising a matrix selected from silica, glass, insoluble synthetic polymers, and insoluble polysaccharides,
   a nucleic acid binding portion for attracting and binding nucleic acids, and
   a cleavable linker portion;
b) combining the solid phase with the sample containing the nucleic acid to bind the nucleic acid to the solid phase;
c) separating the sample from the solid phase;
d) optionally, cleaving the cleavable linker; and
e) releasing the nucleic acid from the solid phase by contacting the solid phase with a reagent composition comprising an aqueous solution having a pH of 7-9, 0.1-3 M metal halide salt or acetate salt and a hydrophilic organic co-solvent at 0.1-50%.

In a preferred embodiment of a solid phase having a cleavable linker, the nucleic acid binding portion is a quaternary onium group of the formula QR₂⁺X⁻ or QR₃⁺X⁻ attached on a surface of the matrix wherein the quaternary onium group is selected from ternary sulfonium groups, quaternary ammonium, and phosphonium groups wherein R is selected from C₁-C₂₀ alkyl, aralkyl and aryl groups, and X is an anion.

The step of separating the sample from the solid phase can be accomplished by for example filtration, gravitational settling, decantation, magnetic separation, centrifugation, vacuum aspiration, overpressure of air or other gas as for example forcing a liquid through a porous membrane or filter mat. Components of the sample other than nucleic acids are removed in this step. To the extent that the removal of other components is not complete, additional washes can be performed to assist in their complete removal.

The step of cleaving the cleavable linker involves treatment of the solid phase having nucleic acid bound thereto with a cleaving agent for a period of time sufficient to break a covalent bond in the cleavable linker portion but not to destroy the nucleic acid. The choice of cleaving agent is determined by the nature of the cleavable linker. When the cleavable linker is a hydrolytically cleavable group, the cleaving agent is water or a lower alcohol or a mixture thereof. The cleaving agent preferably contains a base which when added to water raises the pH.

When the cleavable linker is a reductively cleavable group such as a disulfide or peroxide group the cleaving agent is a reducing agent selected from thiols, amines and phosphines. Exemplary reducing agents include ethanethiol, 2-mercaptoethanol, dithiothreitol, trialkylamine and triphenylphosphine.

Photochemically cleavable linker groups require the use of light as the cleaving agent, typically light in the ultraviolet region or the visible region.

Enzymatically cleavable linker groups as described above are cleaved by enzymes selected from esterases, hydrolases, proteases, peptidases, peroxidases and glycosidases.

When the cleavable linker group is a triggerable dioxetane, the cleaving agent acts to cleave the O—Y bond in the triggering OY group as explained above. Cleaving the O—Y bond destabilizes the dioxetane ring group and leads to fragmentation of the dioxetane ring into two portions by rupture of the C—C and O—O bonds. Triggering agents include an organic or inorganic base, fluoride ion, enzymes, a chemical agent for hydrolyzing an ester, and hydrogen peroxide.

When the cleavable linker is an electron-rich C—C double bond substituted with at least one O, S, or N atom, the cleaving agent is singlet oxygen. Reaction of the double bond group with singlet oxygen produces an unstable 1,2-dioxetane group which spontaneously fragments at ambient temperatures or above. The singlet oxygen can be generated by dye-sensitization or by thermolysis of triphenylphosphite ozonide or anthracene endoperoxides according to methods known in the art of singlet oxygenations.

When the cleavable linker is a ketene dithioacetal as described above, the cleaving agent is a peroxidase enzyme and hydrogen peroxide.

When the cleavable linker is cleaved by a Wittig reaction with a ketone or aldehyde, preferred bases for forming the ylide are alkoxide salts and hydride salts, especially the alkali metal salts. Preferred carbonyl compounds for reaction with the ylide are aliphatic and aromatic aldehydes and aliphatic and aromatic ketones. Acetone is most preferred. Preferred solvents are aprotic organic solvent which can dissolve the reactants and do not consume the base including THF, diethyl ether, p-dioxane, DMF and DMSO.

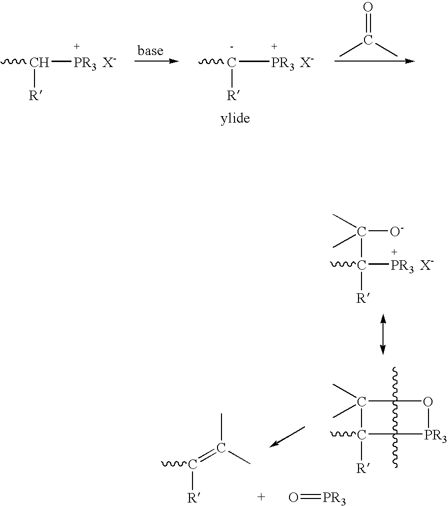

Particularly surprising was the discovery that nucleic acid bound to solid supports of the present invention having as the cleavable linker an alkylene group of at least one carbon atom bonded to either a trialkyl or triarylphosphonium group, (i.e. those solid supports whereby cleavage is accomplished by a Wittig reaction with a ketone or aldehyde) or to a trialkylammonium group, can be made to release the nucleic acid by contact with the novel reagent compositions of the present invention. This result was unexpected since bound nucleic acid is not removed from these solid phase binding materials through contact with numerous other reagents and compositions known in the prior art to elute bound nucleic acids such as
deionized water H₂O
1 M phosphate buffer, pH 6.7
0.1% sodium dodecyl sulfate
0.1% sodium dodecyl phosphate
3 M potassium acetate, pH 5.5
TE (tris EDTA) buffer
50 mM tris, pH 8.5+1.25 M NaCl
0.3 M NaOH+1 M NaCl
1 M NaOH or
1 M NaOH+1 M H₂O₂.

The step of releasing the nucleic acid from the solid phase after cleavage in the methods of the present invention comprises eluting with a solution which dissolves and sufficiently preserves the released nucleic acid. The solution is a reagent composition comprising an aqueous buffer solution having a pH of 7-9, 0.1-3 M metal halide or acetate salt and a hydrophilic organic co-solvent at 0.1-50%. Alternatively the solution can comprise a buffer solution having a pH of about 7-9 wherein the buffering component is present in a concentration of at least 0.1 M and further comprising a hydrophilic organic co-solvent at 0.1-50%. More preferably the hydrophilic organic solvent comprises from about 1-20%. Metal halide salts include alkali metal salts, alkaline earth salts. Preferred salts are sodium acetate, NaCl, KCl, and MgCl₂. Hydrophilic organic co-solvents include methanol, ethanol, n-propanol, 2-propanol, t-butanol, ethylene glycol, propylene glycol, glycerol, 2-mercaptoethanol, dithiothreitol, furfuryl alcohol, 2,2,2-trifluoroethanol, acetone, THF, and p-dioxane. The step of releasing the captured nucleic acid can be subsequent to the cleaving step or concurrent with it. In the latter case the cleaving agent can also act as the elution solution.

The cleaving reaction and releasing (elution) steps can each be performed at room temperature, but any temperature above the freezing point of water and below the boiling point of water can be used. Elution temperature does not appear to be critical to the success of the present methods of isolating nucleic acids. Ambient temperature is preferred, but any temperature above the freezing point of water and below the boiling point of water can be used. Elevated temperatures may increase the rate of elution in some cases or permit the use of compositions containing lower amounts of salts or hydrophilic organic co-solvents. The releasing or elution step can be performed once or can be repeated if necessary one or more times to increase the amount of nucleic acid released.

The cleaving reaction and elution steps can be performed as sequential steps using separate and distinct solutions to accomplish each step. Alternatively the cleaving and elution steps can be performed together in the same step. The latter, concurrent, method is preferred when the cleaving reaction conditions utilize reagents which are compatible with downstream uses of the eluted nucleic acid. Examples are cleaving reactions using moderately alkaline reaction buffers and even stronger alkaline solutions of sodium hydroxide. The former, sequential, method may be desirable in instance where the presence of reagents or solvents for the cleaving reaction are incompatible or undesirable with the nucleic acid. An example of this case is the Wittig release chemistry. Use of separate solutions for cleaving and elution is made possible when the cleaving reaction conditions do not substantially release the DNA into solution.

The method can further comprise a step of washing the solid phase having captured nucleic acid bound thereto with a wash solution to remove other components of the sample from the solid phase. These undesirable substances include enzymes, other types of proteins, polysaccharides, lower molecular weight substances, such as lipids and enzyme inhibitors. Nucleic acid captured on a solid phase of the invention by the above method can be used in captured form in a hybridization reaction to hybridize to labeled or unlabeled complementary nucleic acids. The hybridization reactions are useful in diagnostic tests for detecting the presence or amount of captured nucleic acid. The hybridization reactions are also useful in solid phase nucleic acid amplification processes.

The step of separating the sample from the solid phase can be accomplished by filtration, gravitational settling, decantation, magnetic separation, centrifugation, vacuum aspiration, overpressure of air or other gas to force a liquid through a porous membrane or filter mat, for example. Components of the sample other than nucleic acids are removed in this step. To the extent that the removal of other components is not complete, additional washes can be performed to assist in their complete removal.

The elution composition advantageously permits use of the eluted nucleic acid directly in subsequent downstream processes without the need to evaporate the solvent or precipitate the nucleic acid before use.

When using a reagent composition of the present invention as described above to elute nucleic acid, elution temperature does not appear to be critical to the success of the present methods of isolating nucleic acids. Ambient temperature is preferred, but any temperature above the freezing point of water and below the boiling point of water can be used. Elevated temperatures may increase the rate of elution in some cases. In addition it is recognized that different nucleic acids will be eluted with different facility.

Downstream Uses

An important advantage of these reagent compositions is that they are compatible with many downstream molecular biology processes. Nucleic acid eluted into a reagent composition as described above can in many cases be used directly in a further process. Amplification reactions such as PCR, Ligation of Multiple Oligomers (LMO) described in U.S. Pat. No. 5,998,175, and LCR can employ such nucleic acid eluents. Nucleic acid isolated by conventional techniques, especially from bacterial cell culture or from blood samples, employ a precipitation step. Low molecular weight alcohols are added in high volume percent to precipitate nucleic acid from aqueous solutions. The precipitated materials must then be separated, collected and redissolved in a suitable medium before use. These steps can be obviated by elution of nucleic acid from solid phase binding materials of the present invention using the reagent compositions described above.

Samples from which nucleic acids can be isolated by the methods of the present invention comprise an aqueous solution containing one or more nucleic acids and, optionally, other substances. Representative examples include aqueous solutions of nucleic acids, amplification reaction products, and sequencing reaction products. Materials obtained from bacterial cultures, bodily fluids, blood and blood components, tissue extracts, plant materials, and environmental samples are likewise placed in an aqueous, preferably buffered, solution prior to use.

The methods of solid phase nucleic acid capture can be put to numerous uses. As shown in the particular examples below, both single stranded and double stranded nucleic acid can be captured and released. DNA, RNA, and PNA can be captured and released. A first use is in purification of plasmid DNA from bacterial culture. Plasmid DNA is used as a cloning vector to import a section of recombinant DNA containing a particular gene or gene fragment into a host for cloning.

A second use is in purification of amplification products from amplification reactions. These reactions may be thermally cycled between alternating upper and lower temperatures, such as LMO or PCR, or they may be carried out at a single temperature, e.g., nucleic acid sequence-based amplification (NASBA). The reactions can use a variety of amplification reagents and enzymes, including DNA ligases, RNA polymerases and/or reverse transcriptases. Polynucleotide amplification reaction mixtures that may be purified using the methods of the invention include: ligation of multiple oligomers (LMO), self-sustained sequence replication (3SR), strand-displacement amplification (SDA), “branched chain” DNA amplification, ligase chain reaction (LCR), QB replicase amplification (QBR), ligation activated transcription (LAT), nucleic acid sequence-based amplification (NASBA), repair chain reaction (RCR), cycling probe reaction (CPR), and rolling circle amplification (RCA).

A third use is in sequencing reaction cleanup. Dideoxy terminated sequencing reactions produce ladders of polynucleotides resulting from extension of a primer with a mixture of dNTPs and one ddNTP in each of four reaction mixtures. The ddNTP in each is labeled, typically with a different fluorescent dye. Reaction mixtures contain excess dNTPs and labeled ddNTP, polymerase enzyme and cofactors such as ATP. It is desirable to remove the latter materials prior to sequence analysis.

A fourth use is in isolation of DNA from whole blood. DNA is extracted from leucocytes in a commonly used technique. Blood is typically treated to selectively lyse erythrocytes and after a precipitation or centrifugation step, the intact leucocytes are separately lysed to expose the nucleic acid content. Proteins are digested and the DNA obtained is isolated with a solid phase then used for determination of sequence polymorphism, sequence analysis, RFLP analysis, mutation detection or other types of diagnostic assay.

A fifth use is in isolating DNA from mixtures of DNA and RNA. Methods of the present invention involving strongly alkaline elution conditions, especially those using elevated temperatures, can degrade or destroy RNA present while leaving DNA intact. Methods involving strongly alkaline cleavage reactions will act similarly.

Additional uses include extraction of nucleic acid material from other samples—soil, plant, bacteria, and waste water and long term storage of nucleic acid materials for archival purposes.

Thus a further aspect of the invention comprises methods of isolating and purifying nucleic acids using solid phase binding materials. In one embodiment there is provided a method of isolating a nucleic acid from a sample comprising:
a) providing a solid phase comprising:
   a solid support portion comprising a matrix selected from silica, glass, insoluble synthetic polymers, and insoluble polysaccharides,
   a nucleic acid binding portion for attracting and binding nucleic acids;
b) combining the solid phase with the sample containing the nucleic acid to bind the nucleic acid to the solid phase;
c) separating the sample from the solid phase;
d) releasing the nucleic acid from the solid phase into a solution by contacting the solid phase with a reagent composition comprising an aqueous buffer solution having a pH of 7-9, wherein the concentration of the buffer is at least 0.1 M, and a hydrophilic organic co-solvent at 0.1-50%; and
e) using the solution containing the released nucleic acid directly in a downstream process.

It is a preferred practice to use the solution containing the released nucleic acid directly in a nucleic acid amplification reaction whereby the amount of the nucleic acid or a segment thereof is amplified using a polymerase or ligase-mediated reaction.

### EXAMPLES

Structure drawings when present in the examples below are intended to illustrate only the cleavable linker portion of the solid phase materials. The drawings do not represent a full definition of the solid phase material.

### Example 1

### Synthesis of a Polystyrene Polymer Containing Tributylphosphonium Groups

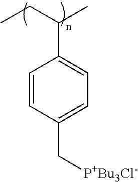

Merrifield peptide resin (Sigma, 1.1 meq/g, 20.0 g) which is a crosslinked chloromethylated polystyrene was stirred in 200 mL of CH₂Cl₂/DMF (50/50) under an argon pad. An excess of tributylphosphine (48.1 g, 10 equivalents) was added and the slurry was stirred at room temperature for 7 days. The slurry was filtered and the resulting solids were washed twice with 200 mL of CH₂Cl₂. The resin was dried under vacuum (21.5 g). Elemental Analysis: Found P, 2.52%; Cl, 3.08%. Expected P, 2.79%; Cl, 3.19%. P/Cl ratio is 0.94.

### Example 2

### Synthesis of a Polystyrene Polymer Containing Trioctylphosphonium Groups

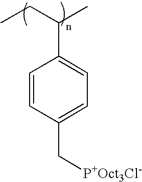

Merrifield peptide resin (Sigma, 1.1 meq/g, 20.0 g) was stirred in 200 mL of CH₂Cl₂/DMF (50/50) under an argon pad. An excess of trioctylphosphine (92.4 g, 10 equivalents) was added and the slurry was stirred at room temperature for 7 days. The slurry was filtered and the resulting solids were washed 3 times with 200 mL of CH₂Cl₂. The resin was dried under vacuum (21.3 g). Elemental Analysis: Found P, 2.28%; Cl, 2.77%. Expected P, 2.77%; Cl, 2.42%. P/Cl ratio is 0.94.

### Example 3

### Synthesis of a Polystyrene Polymer Containing Trimethylphosphonium Groups

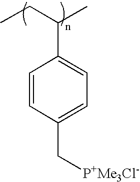

Merrifield peptide resin (ICN Biomedical, 1.6 meq/g, 5.0 g) was stirred in 50 mL of CH₂Cl₂ under an argon pad. A 1.0 M solution of trimethyl phosphine in THF (Aldrich, 12 mL) was added and the slurry was stirred at room temperature for 7 days. An additional 100 mL of CH₂Cl₂ and 1.2 mL of the 1.0 M solution of trimethyl phosphine in THF was added and the slurry was stirred for 3 days. The slurry was filtered and the resulting solids were washed with 125 mL of CH₂Cl₂ followed by 375 mL of methanol. The resin was dried under vacuum (5 g). The resin was ground to a fine powder prior to use.

### Example 4

### Synthesis of a Polystyrene Polymer Containing Triphenylphosphonium Groups

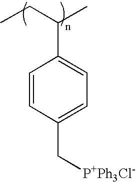

Merrifield peptide resin (ICN Biomedical, 1.6 meq/g, 5.0 g) was stirred in 40 mL of CH₂Cl₂ under an argon pad. Triphenyl phosphine (Aldrich, 3.2 g) was added and the slurry was stirred at room temperature for 5 days. The slurry was filtered and the resulting solids were washed sequentially with CH₂Cl₂₁ MeOH, and CH₂Cl₂. The resin was dried under vacuum (5.4 g).

### Example 5

### Synthesis of a Polystyrene Polymer Containing Tributylammonium Groups

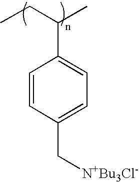

Merrifield peptide resin (Aldrich, 1.43 meq/g, 25.1 g) was stirred in 150 mL of CH₂Cl₂ under an argon pad. An excess of tributyl amine (25.6 g, 4 equivalents) was added and the slurry was stirred at room temperature for 8 days. The slurry was filtered and the resulting solids were washed twice with 250 mL of CH₂Cl₂. The resin was dried under vacuum (28.9 g). Elemental Analysis: Found N, 1.18%; Cl, 3.40%. Expected N, 1.58%; Cl, 4.01%. N/Cl ratio is 0.88.

### Example 6

### Synthesis of a Polystyrene Polymer Containing 2-(tributylphosphonium)acetyl Groups

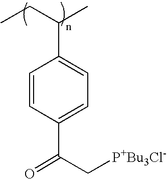

Chloroacetyl polystyrene beads (Advanced Chemtech, 3.0 g, 3.4 meq/g) was added to a solution of tributyl phosphine (4.1 g, 2 equivalents) in 50 mL of CH₂Cl₂ under an argon pad. The slurry was stirred for one week. The slurry was filtered and the resulting solids were washed sequentially with CH₂Cl₂ (4×25 mL), MeOH (2×25 mL), and acetone (4×25 mL). The resin was then air dried.

### Example 7

### Synthesis of Magnetic Particle Having a Polymeric Layer Containing Polyvinylbenzyl Tributylphosphonium Groups

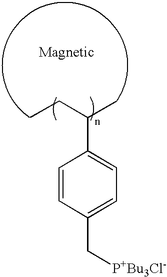

Magnetic Merrifield peptide resin (Chemicell, SiMag Chloromethyl, 100 mg) was added to 2 mL of CH₂Cl₂ in a glass vial. Tributylphosphine (80 μL) was added and the slurry was shaken at room temperature for 3 days. A magnet was placed under the vial and the supernatant was removed with a pipet. The solids were washed four times with 2 mL of CH₂Cl₂ (the washes were also removed by the magnet/pipet procedure). The resin was air dried (93 mg).

### Example 8-Br

### Synthesis of Polymethacrylate Polymer Containing Tributylphosphonium Groups and Bromide Anion

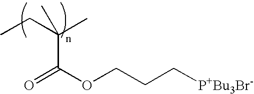

Polymethacrylic acid resin was refluxed with 35 mL of SOCl₂ for 4 h to form the acid chloride. Polymethacryloyl chloride resin (4.8 g) and triethylamine (11.1 g) were stirred in 100 mL of CH₂Cl₂ in an ice water bath under argon. 3-Bromopropanol (9.0 g) was added and the ice water bath was removed. The slurry was stirred overnight at room temperature. The slurry was filtered and the resin was washed 3 times with 40 mL of CH₂Cl₂. The resin was air dried (8.7 g).

The resin (8.5 g) was resuspended and stirred in 100 mL of CH₂Cl₂ under argon. Tributyl phosphine (16.2 g) was added and the slurry stirred for 7 days. The slurry was filtered and the resin was washed 3 times with 100 mL of CH₂Cl₂. The resin was then air dried (5.0 g).

### Example 8-Cl

### Synthesis of Polymethacrylate Polymer Containing Tributylphosphonium Groups and Chloride Anion

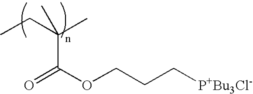

Polymethacryloyl chloride resin (12.2 g) and triethylamine (23.2 g) were stirred in 100 mL of CH₂Cl₂ in an ice water bath under argon. 3-Chloropropanol (12.8 g) was added and the ice water bath was removed. The slurry was stirred overnight at room temperature. The slurry was filtered and the resin was washed 3 times with 100 mL of CH₂Cl₂. The resin was air dried (12.8 g).

The resin (12.8 g) was resuspended and stirred in 100 mL of CH₂Cl₂ under argon. Tributyl phosphine (27.8 g) was added and the slurry stirred for 7 days. The slurry was filtered and the resin was washed with 2×100 mL of CH₂Cl₂ and 2×100 mL of MeOH. The resin was air dried (9.8 g).

### Example 8-S

### Synthesis of Polymethacrylate Polymer Containing Tributylphosphonium Groups and Alkylthioester linkage

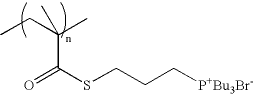

Polymethacryloyl chloride resin (3.6 g) and triethylamine (8.9 g) were stirred in 20 mL of CH₂Cl₂ in an ice water bath under argon. 3-Mercapto-1-propanol (5.8 g), diluted in 20 mL of CH₂Cl₂₁ was added and the ice water bath was removed. The slurry was stirred overnight at room temperature. The slurry was filtered and the resin was washed with CH₂Cl₂₁ water, and methanol. The resin was air dried (3.5 g).

The resin (4.3 g) was resuspended and stirred in 100 mL of dry acetonitrile under argon. Carbon tetrabromide (14.9 g) and triphenyl phosphine (11.8 g) were added. The mixture was refluxed for 5 hours. The slurry was filtered and the resin was washed with 125 mL of acetonitrile, 250 mL of MeOH, and 250 mL of CH₂Cl₂. The resin was then air dried (4.2 g).

The resin (4.2 g) was resuspended and stirred in 40 mL of CH₂Cl₂ under argon. Tributyl phosphine (6.7 g) was added and the slurry stirred for 8 days. The slurry was filtered and the resin was washed with 90 mL of CH₂Cl₂ followed by 50 mL of MeOH. The resin was then air dried (4.0 g).

### Example 9

### Synthesis of Polyvinylbenzyl Polymer Containing Tributylphosphonium Groups and Ester Linkage

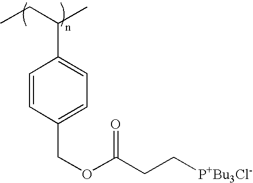

Polystyrene hydroxymethyl acrylate resin (5.0 g) was stirred in 50 mL of acetonitrile in an ice water bath under argon. Tributyl phosphine (2.1 g) and 4.0 M HCl (2.5 mL) were stirred under argon for 15 minutes. This solution was added in 4 equal portions to the resin slurry over 1 hour. The ice water bath was removed and the slurry was stirred at room temperature for 3 hours. The resin was filtered and washed with 50 mL of acetonitrile followed by two 50-mL portions of CH₂Cl₂. The resin was then air dried (6.24 g).

### Example 10

### Synthesis of Polyvinylbenzyl Polymer Containing Tributylphosphonium Groups and Ester Linkage

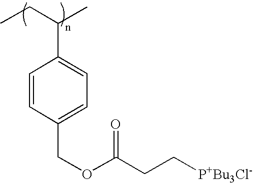

Hydroxymethylated polystyrene (Aldrich, 2.0 meq/g, 5.0 g) and triethylamine (2.3 g) were stirred in 100 mL of CH₂Cl₂ in an ice water bath under argon. Chloroacetyl chloride (1.9 g) was added and the ice water bath was removed. The slurry was stirred overnight at room temperature. The slurry was filtered and the resin was washed 3 times with 40 mL of CH₂Cl₂. The resin was air dried (5.8 g).

The resin (5.8 g) was resuspended and stirred in 100 mL of CH₂Cl₂ under argon. Tributyl phosphine (3.2 g) was added and the slurry stirred for 7 days. The slurry was filtered and the resin was washed 2 times with 100 mL of CH₂Cl₂. The resin was then air dried (5.9 g).

### Example 11

### Synthesis of Polymethacrylate Polymer Containing Tributylphosphonium Groups and Arylthioester Linkage

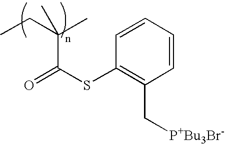

Polymethacryloyl chloride resin (2.7 g) and triethylamine (8.6 g) were stirred in 25 mL of CH₂Cl₂ in an ice water bath under argon. 2-Mercaptobenzyl alcohol (5.0 g), diluted in 20 mL of CH₂Cl₂₁ was added and the ice water bath was removed. The slurry was stirred for 2 days at room temperature. The slurry was diluted with 50 mL of CH₂Cl₂ and centrifuged for 10 minutes at 6000 rpm. The supernatant was discarded. The resin was washed 3 times with 100 mL of MeOH (each wash was centrifuged for 10 minutes at 6000 rpm). After the last wash, the resin was filtered and air dried (4.2 g).

The resin (3.4 g) was resuspended and stirred in 100 mL of dry acetonitrile under argon. Carbon tetrabromide (10.2 g) and triphenyl phosphine (8.0 g) were added. The mixture was refluxed for 4 hours. The slurry was filtered and the resin was washed with 125 mL of acetonitrile, 250 mL of MeOH, and 250 mL of CH₂Cl₂. The resin was then air dried (2.8 g).

The resin (2.8 g) was resuspended and stirred in 40 mL of CH₂Cl₂ under argon. Tributyl phosphine (4.0 g) was added and the slurry stirred for 8 days. The slurry was filtered and the resin was washed with 50 mL of CH₂Cl₂ followed by 125 mL of MeOH. The resin was then air dried (2.7 g).

### Example 12

### Synthesis of Polymethacrylate Polymer Containing Trimethylphosphonium Groups and Arylthioester Linkage

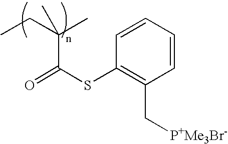

Polymethacryloyl chloride resin (5.1 g) and triethylamine (12.3 g) were stirred in 100 mL of CH₂Cl₂ under argon. 2-Mercaptobenzyl alcohol (9.3 g) was added and the slurry stirred for 5 days at room temperature. The slurry was filtered and the resin was washed with 300 mL of CH₂Cl₂, 500 mL of water, and 200 mL of MeOH. The resin was air dried (5.8 g).

The resin (4.8 g) was resuspended and stirred in 100 mL of dry acetonitrile under argon. Carbon tetrabromide (14.3 g) and triphenyl phosphine (11.3 g) were added. The mixture was refluxed for 4 hours. The slurry was filtered and the resin was washed with 100 mL of acetonitrile, 200 mL of CH₂Cl₂, 200 mL of MeOH, and 200 mL of CH₂Cl₂. The resin was then air dried (4.8 g).

The resin (1.04 g) was resuspended and stirred in 30 mL of CH₂Cl₂ under argon. A 1.0 M solution of trimethyl phosphine in THF (7.3 mL) was added and the slurry stirred for 10 days. The slurry was filtered and the resin was washed with 100 mL of CH₂Cl₂₁ 100 mL of THF, 50 mL of MeOH, and 100 mL of CH₂Cl₂. The resin was then air dried (1.1 g).

### Example 13

### Synthesis of Polymethacrylate Polymer Containing Trioctylphosphonium Groups and Arylthioester Linkage

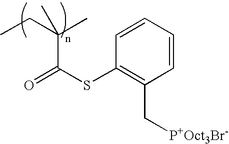

Polymethacryloyl chloride resin (5.1 g) and triethylamine (12.3 g) were stirred in 100 mL of CH₂Cl₂ under argon. 2-Mercaptobenzyl alcohol (9.3 g) was added and the slurry stirred for 5 days at room temperature. The slurry was filtered and the resin was washed with 300 mL of CH₂Cl₂, 500 mL of water, and 200 mL of MeOH. The resin was air dried (5.8 g).

The resin (4.8 g) was resuspended and stirred in 100 mL of dry acetonitrile under argon. Carbon tetrabromide (14.3 g) and triphenyl phosphine (11.3 g) were added. The mixture was refluxed for 4 hours. The slurry was filtered and the resin was washed with 100 mL of acetonitrile, 200 mL of CH₂Cl₂, 200 mL of MeOH, and 200 mL of CH₂Cl₂. The resin was then air dried (4.8 g).

The resin (1.68 g) was resuspended and stirred in 30 mL of CH₂Cl₂ under argon. Trioctyl phosphine (4.4 g) was added and the slurry stirred for 10 days. The slurry was filtered and the resin was washed with 100 mL of CH₂Cl₂₁ 100 mL of THF, 50 mL of MeOH, and 100 mL of CH₂Cl₂. The resin was then air dried (1.67 g).

### Example 14

### Synthesis of Magnetic Silica Particles Functionalized with Polymethacrylate Linker and Containing Tributylphosphonium Groups and Arylthioester Linkage

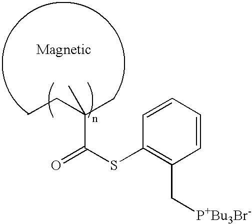

Magnetic carboxylic acid-functionalized silica particles (Chemicell, SiMAG-TCL, 1.0 meq/g, 0.6 g) were placed in 6 mL of thionyl chloride and refluxed for 3 hours. The excess thionyl chloride was removed under reduced pressure. The resin was resuspended in 40 mL of CH₂Cl₂ in an ice water bath under argon. Triethylamine (1.2 g) was added. 2-Mercaptobenzyl alcohol (0.7 g) was added and the ice water bath was removed. The slurry was shaken overnight at room temperature. The slurry was filtered and the resin was centrifuged twice with 35 mL of MeOH at 5000 rpm for 10 minutes. The supernatants were discarded. The orange-yellow resin was air dried (335 mg).

The resin (335 mg) was resuspended in 45 mL of dry acetonitrile under argon. Carbon tetrabromide (2.0 g) and triphenylphosphine (1.6 g) were added. The mixture was refluxed for 3 hours. The resin was centrifuged at 5000 rpm for 10 minutes and the supernatant was discarded. The resin was centrifuged twice with 50 mL of acetonitrile at 5000 rpm for 10 minutes and the supernatants were discarded. The resin was then air dried (328 mg).

The resin (328 mg) was resuspended in 40 mL of CH₂Cl₂ under argon. Tributylphosphine (280 mg) was added and the slurry shaken for 10 days. The magnetic resin settled by placing a magnet on the exterior of the flask and the supernatant was decanted. The resin was washed 3 times with 30 mL of CH₂Cl₂ followed with 3 washes of 25 mL of MeOH. The resin was then air dried (328 mg).

### Example 15

### Synthesis of Magnetic Polymeric Methacrylate Particles Containing Tributylphosphonium Groups and Arylthioester Linkage

Sera-Mag™ Magnetic Carboxylate Microparticles (Seradyn) were used to form cleavable magnetic particles. The Sera-Mag particles comprise a polystyrene-acrylic acid polymer core surrounded by a magnetite coating encapsulated with proprietary polymers. Carboxylate groups are accessible on the surface. Particles (0.52 meq/g, 0.50 g) were suspended in 15 mL of water and 25 mL of 0.1 M MES buffer (pH 4.0). The reaction mixture was sonicated for 5 minutes prior to the addition of 126 mg of EDC (1-[3-(dimethylamino)propyl]-3-ethyl carbodiimide hydrochloride) and 110 mg of 2-mercaptobenzyl alcohol. The reaction mixture was shaken for 7 days. The reaction mixture was filtered. The resin was washed with 50 mL of water and 100 mL of MeOH. The resin was air dried (0.53 g).

The resin (0.53 g) was resuspended in 20 mL of dry acetonitrile under argon. Carbon tetrabromide (174 mg) and triphenyl phosphine (138 mg) were added. The mixture was sonicated at 65° C. for 5 hours. The reaction mixture was placed on a large magnet and the supernatant was decanted. The resin was washed 4 times with acetonitrile, the resin was precipitated by a magnet, and the washes were discarded. The resin was resuspended in MeOH and shaken overnight. The resin was washed 4 times with MeOH, the resin was precipitated by a magnet, and the washes were discarded. The resin was then air dried (0.52 g).

The resin (0.52 g) was resuspended in 10 mL of acetonitrile. Tributylphosphine (106 mg) was added and the reaction shaken for 7 days. The magnetic resin was precipitated by a magnet and the supernatant was decanted. The resin was washed 4 times with acetonitrile and 4 times with MeOH. The resin was then air dried (0.51 g).

### Example 16

### Synthesis of Polymethacrylate Polymer Containing Tributylphosphonium Groups and Arylthioester Linkage

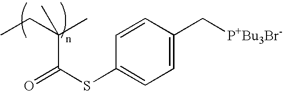

Polymethacryloyl chloride resin (0.6 g) and triethylamine (1.5 g) were stirred in 30 mL of CH₂Cl₂ in an ice water bath under argon. 4-Mercaptobenzyl alcohol (1.0 g), diluted in 20 mL of CH₂Cl₂₁ was added and the ice water bath was removed. The slurry was stirred for 2 days at room temperature. The slurry was filtered and washed with 50 mL of CH₂Cl₂₁ 100 mL of water, 50 mL of MeOH, and 25 mL of CH₂Cl₂. The resin was air dried (0.7 g).

The resin (0.6 g) was resuspended and stirred in 20 mL of dry acetonitrile under argon. Carbon tetrabromide (1.8 g) and triphenylphosphine (1.4 g) were added. The mixture was refluxed for 3 hours. The slurry was filtered and the resin was washed with acetonitrile, MeOH, and CH₂Cl₂. The resin was then air dried (0.6 g).

The resin (0.6 g) was resuspended and stirred in 15 mL of CH₂Cl₂ under argon. Tributylphosphine (0.85 g) was added and the slurry stirred for 6 days. The slurry was filtered and the resin was washed with 75 mL of CH₂Cl₂ followed by 150 mL of MeOH. The resin was then air dried (0.6 g).

### Example 17

### Synthesis of Polymethacrylate Polymer Containing Tributylphosphonium Groups and Arylthioester Linkage

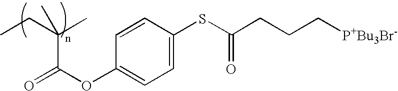

Polymethacryloyl chloride resin (0.71 g) and triethylamine (2.2 g) were stirred in 100 mL of CH₂Cl₂ under argon. 4-Hydroxyphenyl 4-bromothiobutyrate (2.55 g) was added and the slurry was stirred for 5 days at room temperature. The slurry was filtered and washed with CH₂Cl₂ and hexanes. The resin was air dried (0.85 g).

The resin (0.85 g) was resuspended and stirred in 20 mL of CH₂Cl₂ under argon. Tributylphosphine (2.7 g) was added and the slurry stirred for 3 days. The slurry was filtered and the resin was washed with CH₂Cl₂ and hexanes. The resin was then air dried.

### Example 18

### Synthesis of Polymethacrylate Polymer Containing Tributylphosphonium Groups and Arylthioester Linkage

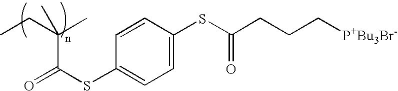

Polymethacryloyl chloride resin (1.0 g) and pyridine (1.9 mL) were stirred in 20 mL of CH₂Cl₂ under argon. 1,4-Benzene dithiol (1.85 g) was added and the slurry was stirred overnight at room temperature. The slurry was filtered and washed with CH₂Cl₂ and hexanes. The resin was air dried (1.08 g).

The resin (1.08 g) and triethylamine (3.0 mL) were stirred in 20 mL of CH₂Cl₂ under argon. 4-Bromobutyryl chloride (1.8 mL) was added and the reaction mixture was stirred for 2 days. The slurry was filtered and washed with CH₂Cl₂. The resin was air dried (1.10 g).

The resin (1.10 g) was resuspended and stirred in 30 mL of CH₂Cl₂ under argon. Tributylphosphine (4.0 g) was added and the slurry stirred for 5 days. The slurry was filtered and the resin was washed with CH₂Cl₂. The resin was then air dried (1.0 g).

### Example 19

### Synthesis of Crosslinked Polystyrene Polyethylene Glycol Succinate Copolymer Containing Tributylphosphonium Groups

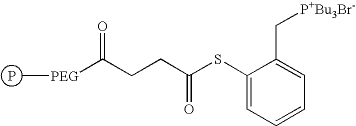

TentaGel S COOH beads (Advanced Chemtech, 3.0 g), a crosslinked polystyrene polyethylene glycol succinate copolymer, were refluxed in 30 mL of thionyl chloride for 90 minutes. The residual thionyl chloride was removed under reduced pressure. The resin was resuspended in 30 mL of chloroform and reconcentrated.

The resin and triethylamine (0.14 g) were stirred in 60 mL of CH₂Cl₂ in an ice water bath under argon. 2-Mercaptobenzyl alcohol (0.11 g) was added and the ice water bath was removed. The slurry was stirred for 2 days at room temperature. The slurry was filtered and the resin was washed with CH₂Cl₂₁ water, MeOH, and CH₂Cl₂. The resin was filtered and air dried (2.9 g).

The resin (2.8 g) was resuspended and stirred in 60 mL of dry acetonitrile under argon. Carbon tetrabromide (0.36 g) and triphenylphosphine (0.29 g) were added. The mixture was refluxed for 4 hours. The slurry was filtered and the resin was washed with acetonitrile, MeOH, and CH₂Cl₂. The resin was then air dried (2.8 g).

The resin (2.7 g) was resuspended and stirred in 50 mL of CH₂Cl₂ under argon. Tributylphosphine (0.21 g) was added and the slurry stirred for 6 days. The slurry was filtered and the resin was washed with 50 mL of CH₂Cl₂ followed by 175 mL of MeOH. The resin was then air dried (2.8 g).

### Example 20

### Synthesis of Controlled Pore Glass Beads Containing Succinate-Linked Tributylphosphonium Groups and a Thioester Linkage

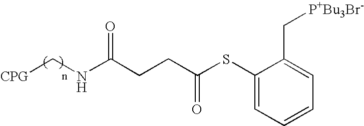

Millipore LCAA glass (1.0 g, 38.5 μmole/gram) was suspended in 10 mL of dry pyridine. Succinic anhydride (40 mg) was added and the reaction mixture was shaken at room temperature for 4 days. The reaction mixture was diluted with 20 mL of MeOH and the mixture was filtered. The solids were washed 5 times with 20 mL of MeOH and 5 times with 20 mL of CH₂Cl₂. The solids were air dried (1.0 g).

The solids (0.50 g) were suspended in 10 mL of dry CH₂Cl₂. Dicyclohexylcarbodiimide (10 mg) and 2-mercaptobenzyl alcohol were added and the reaction mixture was shaken at room temperature for 6 days. The reaction mixture was diluted with CH₂Cl₂ and the mixture was filtered. The solids were washed 3 times with MeOH and 3 times with CH₂Cl₂. The solids were air dried (0.50 g).

The solids (400 mg) were resuspended in 10 mL of dry acetonitrile under argon. Carbon tetrabromide (14 mg) and triphenylphosphine (11 mg) were added. The mixture was refluxed for 3 hours. The mixture was filtered and the solid was washed 5 times with 50 mL of MeOH and 5 times with 50 mL of CH₂Cl₂. The solids were air dried (360 mg).

The solid (300 mg) was resuspended in 10 mL of CH₂Cl₂ under argon. Tributylphosphine (5 drops) was added and the reaction mixture was shaken for 5 days. The reaction mixture was diluted with CH₂Cl₂ and filtered. The solid was washed 5 times with 50 mL of CH₂Cl₂ and air dried (300 mg).

### Example 21

### Synthesis of Polyvinylbenzyl Polymer Containing Acridinium Ester Groups

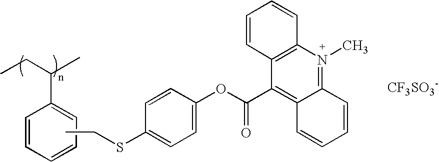

Acridine 9-carboxylic acid chloride, 1.25 g) and triethylamine (1.3 g) were stirred in 40 mL of CH₂Cl₂ in an ice water bath under argon. Hydroxythiophenol resin (Polymer Laboratories, 1.67 meq/g, 3.0 g) was added and the ice water bath was removed. The slurry was stirred overnight at room temperature. The slurry was filtered and the resin was washed 3 times with 200 mL of CH₂Cl₂. The resin was air dried (4.4 g).

The resin (4.3 g) was stirred in 40 mL of CH₂Cl₂ under argon. Methyl triflate (6.1 g) was added and the reaction mixture was stirred for 2 days. The slurry was filtered and the resin was washed with 200 mL of CH₂Cl₂ and 1 L of MeOH. The resin was vacuum-dried (4.7 g).

### Example 22

### Synthesis of Polyvinylbenzyl Polymer Containing Acridan Ketene Dithioacetal Groups

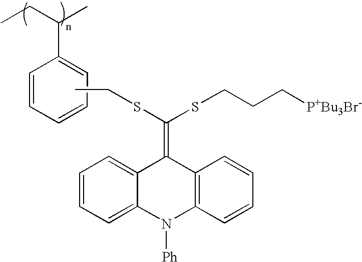

N-Phenyl acridan (0.62 g) was stirred in 20 mL of anhydrous THF at −78° C. under argon. Butyl lithium (2.5 M in hexanes, 0.93 mL) was added and the reaction mixture stirred at −78° C. for 2 hours. Carbon disulfide (0.16 mL) was added and the reaction mixture was stirred at −78° C. for 1 hour. The reaction mixture was warmed to room temperature. Merrifield peptide resin (1.6 meq/g, 1.0 g) was added and the mixture stirred at room temperature overnight. The mixture was filtered. The resin was washed 5 times with 10 mL of acetone, 3 times with 10 mL of water, and twice with 10 mL of acetone. The resin was air dried (1.21 g).

The resin (1.21 g) and NaH (60% in oil, 0.003 g) were stirred in 20 mL of anhydrous DMF under argon for 4 hours. 1,3-Dibromopropane (0.07 mL) was added and the mixture stirred for 3 days. The mixture was filtered. The resin was washed 3 times with 10 mL of acetone, 5 times with 10 mL of water, and 5 times with 10 mL of acetone. The resin was air dried (1.22 g).

The resin (1.22 g) was resuspended and stirred in 20 mL of DMF under argon. Tributylphosphine (1.18 g) was added and the slurry stirred for 7 days. The slurry was filtered and the resin was washed 4 times with 20 mL of CH₂Cl₂ and 4 times with 20 mL of acetone. The resin was then air dried (1.07 g).

### Example 23

### General Procedure for Binding and Eluting DNA from Hydrolytically Cleavable Particles

A 10 mg sample of beads was rinsed with 500 μL of THF in a tube. The contents were centrifuged and the liquid removed. The rinse process was repeated with 200 μL of water. A solution of 2 μg of linearized pUC18 DNA in 200 μL of water was added to the beads and the mixture gently shaken for 20 min. The mixture was spun down and the supernatant collected. The beads were rinsed with 2×200 μL of water and the water discarded. DNA was eluted by incubating the beads with 200 μL of aq. NaOH at 37° C. for 5 min. The mixture was spun down and the eluent removed for analysis.

### Example 24

### Fluorescent Assay Protocol

Supernatants and eluents were analyzed for DNA content by a fluorescent assay using PicoGreen™ to stain DNA. Briefly, 10 μL aliquots of solutions containing or suspected to contain DNA are incubated with 190 μL of a fluorescent DNA “staining” solution. The fluorescent stain was PicoGreen (Molecular Probes) diluted 1:400 in 0.1 M tris, pH 7.5, 1 mM EDTA. Fluorescence was measured in a microplate fluorometer (Fluoroskan, Labsystems) after incubating samples for at least 5 min. The filter set was 480 nm and 535 nm. Positive controls containing a known amount of the same DNA and negative controls were run concurrently.

### Example 25

### Binding DNA onto Beads of Example 11 from Different pH Solutions Showing Effective Capture Over a Wide Range of pH

Buffers spanning the pH range 4.5 to 9.0 were prepared. Buffers having pH 4.5 to 6.5 were 10 mM acetate buffers. Buffers having pH 7.0 to 9.0 were 10 mM tris acetate buffers. A solution of 2 μg of linearized pUC18 DNA in 200 μL of each buffer was added to 10 mg of the cleavable beads of example 11 for 30-45 s at room temperature. Negative control solutions with no DNA in each buffer were run in parallel. Supernatants were removed after spinning bead samples down and analyzed by UV and fluorescence.

| Buffer pH | % Bound (by UV) | % Bound (by Fl.) |
|---|---|---|
| 4.5 | 56 | 73 |
| 5.0 | 64 | 68 |
| 5.5 | 58 | 64 |
| 6.0 | 61 | 71 |
| 6.5 | 57 | 74 |
| 7.0 | 49 | 61 |
| 7.5 | 44 | 60 |
| 8.0 | 45 | 55 |
| 8.5 | 37 | 39 |
| 9.0 | 31 | 33 |

Separately it was found that binding for 5 min using 20 mg of beads at pH 8.0 resulted in 100% capture of DNA.

### Example 26

### Use of DNA Eluted from Cleavable Beads of Example 11 in LMO Amplification

Solutions containing 0.1 or 1 μg of pUC18 DNA in 200 μL of water were added to 10 mg of beads previously washed with 400 μL of THF and then twice with water. After incubation for 30 min the sample tubes were spun down for 30 s and the supernatants collected. The beads were washed with 2×400 μL of water and the washes discarded. DNA was eluted by washing the beads with 100 μL of 1 M NaOH at room temperature for 15 min, spinning for 30 s and collecting the eluent. An 80 μL portion of each eluent was neutralized with 40 μL of 1 M acetic acid.

Plasmid DNA isolated using the polymeric beads of the invention was amplified by LMO as described in U.S. Pat. No. 5,998,175 using the eluent directly without precipitating the DNA. Briefly, a 68 bp region was amplified by a thermocycling protocol using a pair of primers and a set of octamers spanning the 68 base region. A set of twelve octamer-5′-phosphates (six per strand), the primers and template (1 μL) were dissolved in Ampligase buffer. Reaction tubes were overlaid with 50 μL of mineral oil and heated to 94° C. for 5 min. After about 2 min 100 U of Ampligase was added to each tube. Samples were cycled 35 times at 94° C. for 30 s; 55° C. for 30 s; 35° C. for 30 s. Gel electrophoresis of the amplification reactions revealed a band of the expected molecular weight.

### Example 27

### Binding of DNA to Polymer Beads of Example 9

A 100 mg sample of beads was rinsed with 1 mL of THF in a tube. The contents were centrifuged and the liquid removed. The rinse process was repeated twice with 1 mL of water. A solution of 80 μg of pUC18 DNA in 1 mL of water was added to the beads and the mixture gently shaken for 20 min. The mixture was spun down and the supernatant collected for UV analysis. The supernatant contained 66 μg of DNA. The binding capacity was thus determined to be 0.14 μg/mg.

### Example 28

### Binding and Release of RNA from Cleavable Beads of Example 11

In two tubes, 2 μg of Luciferase RNA was bound to 10 mg of beads. 1× Reverse transcriptase buffer (50 mM tris-HCl, pH 8.5, 8 mM MgCl₂, 30 mM KCl, 1 mM DTT (0.015%)) was used for elution. One tube was heated for 5 min at 94° C. and the other tube was heated for 30 min at 94° C. The eluents and controls were run on a 1% agarose gel and stained with SYBR Greene. The 5 min heating showed ˜50% elution of RNA from the beads but the 30 min heating seemed to denature RNA.

### Example 29

### Binding and Release of RNA from Cleavable Beads of Example 11 with Different Cleavage/Elution Buffers

In three tubes, 1 μg of Luciferase RNA was bound to 10 mg of beads. In one tube, 3M potassium acetate was used to elute the RNA at room temperature for 30 min. In another tube, 1× reverse transcriptase buffer (RT) was used for elution at 94° C. for 1 min. The third tube had RNA extraction buffer and was heated to 94° C. for 1 min. RNA extraction buffer consists of 10 mM tris-HCl, pH 8.8, 0.14 M NaCl, 1.5 M MgCl₂, 0.5% NP-40, 1 mM DTT. All eluents and controls were run on a 1% agarose gel and stained with SYBR Green™. The 3M potassium acetate did not produce recognizable RNA. The 1× reverse transcriptase buffer and RNA extraction buffer both showed a band estimated to contain RNA corresponding to about 50% elution.

### Example 30

### Binding and Release of RNA from Cleavable Beads of Example 11 and Detection by Chemiluminescent Blot Assay

In four tubes, 1 μg of Luciferase RNA was bound to 10 mg of beads. Two tubes used the 1× reverse transcriptase buffer for elution and the other two used RNA extraction buffer. One tube of each kind of buffer was heated to 94° C. for 1 min. The other two tubes were heated to 94° C. for 5 min. All eluents and controls were run on a 1% agarose gel and stained with SYBR Green. The eluents heated 1 min contained more RNA than those heated for 5 min using either buffer. RNA extraction buffer eluted more RNA than the 1×RT buffer. The RNA was transferred onto a nylon membrane with an overnight capillary transfer. The RNA was then hybridized overnight with HF-1 biotin labeled primer. Detection was done with anti-biotin HRP and Lumigen PS-3 as chemiluminescent substrate. The 5 min exposure verified the gel results.

### Example 31

### Binding and Release of RNA from Cleavable Beads of Example 11 at Various Temperatures

In six tubes, 1 μg of Luciferase RNA was bound to 10 mg of beads. RNA extraction buffer was used to elute the RNA for 5 min at several different temperatures: 40° C., 50° C., 60° C., 70° C., 80° C., and 90° C. All eluents and controls were run on a 1% agarose gel and stained with SYBR Green. All temperatures appeared to elute 100%.

### Example 32

### Binding of Linearized pUC18 DNA with Tributylphosphonium Beads of Example 1 and Release with Different Elution Compositions

A 10 mg sample of beads was rinsed with 500 μL of THF in a tube. The contents were centrifuged and the liquid removed. The rinse process was repeated with 200 μL of water. A solution of 2 μg of linearized pUC18 DNA in 200 μL of water was added to the beads and the mixture gently shaken for 20 min. The mixture was spun down and the supernatant collected. The beads were rinsed with 2×200 μL of water and the water discarded. DNA was eluted by incubating the beads with 200 μL of various reagent compositions described in the table below at room temperature for 20 min. The mixture was spun down and the eluent removed for fluorescence analysis as described in example 24.

| Buffer | Salt | Org. Solvent | % Eluted |
|---|---|---|---|
| 50 mM tris, pH 8.5 | 1.25 M NaCl | 15% furfuryl | 58 |
| | | alcohol | |
| 50 mM tris, pH 8.5 | 1.25 M NaCl | 15% ficoll | 19 |
| 50 mM tris, pH 8.5 | 1.25 M NaCl | 15% HOCH₂CH₂SH | 52 |
| 50 mM tris, pH 8.5 | 1.25 M NaCl | 15% DTT | 52 |
| 50 mM tris, pH 8.5 | 1.25 M NaCl | 15% glycerol | 15 |
| 50 mM tris, pH 8.5 | 1.25 M NaCl | 15% 2-propanol | 50 |
| 50 mM tris, pH 8.5 | 1.25 M NaCl | 15% ethanol | 37 |
| 50 mM tris, pH 8.5 | 1.25 M NaCl | 15% CF₃CH₂OH | 38 |
| 50 mM tris, pH 8.5 | 1.25 M NaCl | 15% acetone | 42 |
| 50 mM tris, pH 8.5 | 1.25 M NaCl | 15% THF | 41 |
| 50 mM tris, pH 8.5 | 1.25 M NaCl | 15% p-dioxane | 33 |

### Example 33

The bind and release protocol of example 32 was followed with reagent compositions described in the table below. The effect of changing the concentration of either DTT or 2-mercaptoethanol was examined.

| Buffer | Salt | Org. Solvent | % Eluted |
|---|---|---|---|
| 50 mM tris, pH 8.5 | 1.25 M NaCl | 0.1% DTT | 0 |
| 50 mM tris, pH 8.5 | 1.25 M NaCl | 1% DTT | 0 |
| 50 mM tris, pH 8.5 | 1.25 M NaCl | 3% DTT | 36 |
| 50 mM tris, pH 8.5 | 1.25 M NaCl | 4% DTT | 41 |
| 50 mM tris, pH 8.5 | 1.25 M NaCl | 0.1% HOCH₂CH₂SH | 0 |
| 50 mM tris, pH 8.5 | 1.25 M NaCl | 1% HOCH₂CH₂SH | 0 |
| 50 mM tris, pH 8.5 | 1.25 M NaCl | 3% HOCH₂CH₂SH | 39 |
| 50 mM tris, pH 8.5 | 1.25 M NaCl | 4% HOCH₂CH₂SH | 38 |

### Example 34

The bind and release protocol of example 32 was followed with reagent compositions described in the table below. The effect of changing the concentration of salts NaCl and KCl was examined.

| Buffer | Salt | Org. Solvent | % Eluted |
|---|---|---|---|
| 50 mM tris, pH 8.5 | 0.1 M NaCl | 5% DTT | 1 |
| 50 mM tris, pH 8.5 | 0.25 M NaCl | 5% DTT | 0 |
| 50 mM tris, pH 8.5 | 0.5 M NaCl | 5% DTT | 27 |
| 50 mM tris, pH 8.5 | 0.75 M NaCl | 5% DTT | 29 |
| 50 mM tris, pH 8.5 | 1.0 M NaCl | 5% DTT | 29 |
| 50 mM tris, pH 8.5 | 1.25 M NaCl | 5% DTT | 26 |
| 50 mM tris, pH 8.5 | 0.75 M KCl | 5% DTT | 64 |
| 50 mM tris, pH 8.5 | 1.25 M KCl | 5% DTT | 60 |

### Example 35

The bind and release protocol of example 32 was followed with reagent compositions described in the table below. Beads were eluted for 60 min.

| Buffer | Salt | Org. Solvent | % Eluted |
|---|---|---|---|
| 50 mM tris, pH 8.5 | 0.1 M NaCl | 0% 2-propanol | 3 |
| 50 mM tris, pH 8.5 | 0.1 M NaCl | 15% 2-propanol | 68 |
| 50 mM tris, pH 8.5 | 0.25 M NaCl | 30% 2-propanol | 64 |
| 50 mM tris, pH 8.5 | 0.5 M NaCl | 50% 2-propanol | 4 |

### Example 36

The bind and release protocol of example 32 was followed with reagent compositions described in the table below. Relative effectiveness is scored.

| Buffer | Salt | Org. Solvent | |
|---|---|---|---|
| 50 mM tris, pH 8.5 | 1.0 M Na acetate | 15% 2-propanol | ++ |
| 50 mM tris, pH 8.5 | 1.5 M Na acetate | 15% 2-propanol | ++ |
| 50 mM tris, pH 8.5 | 1.25 M Na acetate | 15% 2-propanol | ++ |
| 50 mM tris, pH 8.5 | 0.75 M Na acetate | 15% 2-propanol | + |
| 50 mM tris, pH 8.5 | 0.5 M Na acetate | 15% 2-propanol | + |
| 50 mM tris, pH 8.5 | 0.1 M Na acetate | 15% 2-propanol | + |

### Example 37

### Binding of Oligonucleotides of Different Lengths with Tributylphosphonium Beads of Example 1 and Release with a Reagent Composition

The bind and release protocol of example 32 was performed on various size oligonucleotides ranging from 20 bases to 2.7 kb. The elution composition was 50 mM tris, pH 8.5, 0.75 M NaCl, 5% DTT. The amount of DNA was determined fluorometrically using OliGreen™, a fluorescent stain for ssDNA.

| Oligonucleotide size (nt) | % Eluted |
|---|---|
| 20 | 39 |
| 30 | 43 |
| 50 | 36 |
| 68 | 34 |
| 181 | 33 |
| 424 | 33 |
| 753 | 32 |
| 2.7 kb | 20 |

### Example 38

### Binding of Linearized pUC18 DNA with Tributylphosphonium Beads of Example 1 and Release with Different Elution Volumes

A solution of 2 μg of linearized pUC18 DNA in 200 μL of water was added to 10 mg of beads in a 2 mL spin column (Costar). After incubation for 20 min the column was spun down and the supernatant collected. The beads were washed with 2×200 μL of water and the washes discarded. DNA was eluted by washing the beads with 5×200 μL of 50 mM tris, pH 8.5, 0.75 M NaCl, 5% DTT at room temperature for 5 min, spinning and collecting the eluent for analysis by fluorescence and gel electrophoresis after each elution.

In a similar manner, beads containing bound DNA were eluted with 5×40 μL of the same elution buffer.

Percent Eluted

| | 200 μL elutions | 40 μL elutions |
|---|---|---|
| Elution 1 | 63 | 47 |
| Elution 2 | 10 | 11 |
| Elution 3 | 5.5 | 10 |
| Elution 4 | 3.5 | 5 |
| Elution 5 | 2.1 | 4 |
| Total | 84 | 77 |

### Example 39

### Binding and Release of Nucleic Acid with Tributylammonium Beads of Example 5

A solution of 2 μg of linearized pUC18 DNA in 200 μL of water was added to 10 mg of beads and the mixture gently shaken for 30 min. The mixture was spun down and the supernatant collected. The beads were rinsed with 2×200 μL of water and the water discarded. DNA was eluted by incubating the beads with 200 μL of 50 mM tris, pH 8.5, 0.75 M NaCl, 5% DTT at room temperature for 30 min. The mixture was spun down and the eluent removed for fluorescence analysis as described in example 26. DNA binding was 50%, elution was 69% of the bound portion.

### Example 40

### Binding and Release of Nucleic Acid with Magnetic Tributylphosphonium Beads of Example 7

A 10 mg sample of beads was rinsed with 500 μL of THF in a tube. The contents were magnetically separated and the liquid removed. The rinse process was repeated with 200 μL of water. A solution of 2 μg of linearized pUC18 DNA in 200 μL of water was added to the beads and the mixture gently shaken for 20 min. The mixture was separated magnetically and the supernatant collected. The beads were rinsed with 2×200 μL of water and the water discarded. DNA was eluted by incubating the beads with 200 μL of 50 mM tris, pH 8.5, 1.25 M NaCl, 15% 2-propanol at room temperature for 30 min. The mixture was separated magnetically and the eluent removed for fluorescence analysis as described in example 26. DNA binding was 100%, elution was 18%.

### Example 41

### Binding of Linearized pUC18 DNA with Tributylphosphonium Beads of Example 1 and Release with Different Elution Temperatures

A solution of 2 μg of linearized pUC18 DNA in 200 μL of water was added to 10 mg of beads and the mixture gently shaken for 30 min. The mixture was spun down and the supernatant collected. The beads were rinsed with 2×200 μL of water and the water discarded. DNA was eluted by incubating the beads with 200 μL of 50 mM tris, pH 8.5, 1.25 M NaCl, 15% 2-propanol for 5 min at various temperatures: 37° C., 46° C., 65° C., and 94° C. The mixture was spun down and the eluent removed for fluorescence analysis as described in example 26. DNA binding was 100%, ˜65-70% of the bound DNA was eluted at all temperatures.

### Example 42

### Synthesis of Polymethacrylate Polymer Containing Tributylphosphonium Groups and Arylthioester Linkage

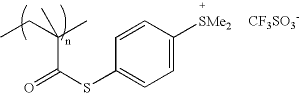

Polymethacryloyl chloride resin, prepared as described above, (2.96 g), 5.07 g of 4-(methylthio)thiophenol and triethylamine (8.8 mL) were stirred in 100 mL of CH₂Cl₂ at room temperature under argon for 5 days. The solid was filtered off and washed with 100 mL of CH₂Cl₂ and 100 mL of water and then was stirred in 125 mL of methanol for several days. Filtration and drying yielded 3.76 g of the thioester product.

A 2.89 g portion of the solid in 100 mL of CH₂Cl₂ was stirred with 4.1 mL of methyl triflate for 7 days. The solid was filtered and washed sequentially with 200 mL of CH₂Cl₂, 300 mL of methanol and 300 mL of CH₂Cl₂ and then air dried.

### Example 43

### Binding and Release of DNA Using Cleavable Beads Having Dimethylsulfonium Group

A solution of 2 μg of linearized pUC18 DNA in 200 μL of 10 mM tris, pH 8 was added to a 10 mg sample of the beads of example 42 and the mixture gently shaken for 5 min. The mixture was spun down and the supernatant collected. The beads were rinsed with 2×200 μL of water and the water discarded. DNA was eluted by incubating with 200 μL of 50 mM tris, pH 8.5, 0.75 M NaCl, 5% DTT at 37° C. for 5 min. The mixture was spun down and the eluent removed for fluorescence analysis. The supernatant contained no DNA. The eluent contained 37% of the initially bound DNA.

### Example 44

### Binding of Linearized pUC18 DNA with Tributylphosphonium Beads of Example 1 and Release with Different Elution Compositions

A 10 mg sample of beads was rinsed with 200 μL of water. A solution of 2 μg of linearized pUC18 DNA in 200 μL of water was added to 10 mg of the beads and the mixture gently shaken for 25 min. The mixture was spun down and the supernatant collected. The beads were rinsed with 2×200 μL of water and the water discarded. DNA was eluted by incubating the beads with 200 μL of various reagent compositions described in the table below at room temperature for 25 min. The mixture was spun down and the eluent removed for analysis by fluorescence and gel electrophoresis.

| Buffer | [MgCl2] | Org. Solvent | % Eluted |
|---|---|---|---|
| 50 mM tris, pH 8.5 | 2.0 M | 5% DTT | 7.3 |
| 50 mM tris, pH 8.5 | 1.5 M | 5% DTT | 10.3 |
| 50 mM tris, pH 8.5 | 1.25 M | 5% DTT | 11.5 |
| 50 mM tris, pH 8.5 | 1.0 M | 5% DTT | 13.3 |
| 50 mM tris, pH 8.5 | 0.75 M | 5% DTT | 17.6 |
| 50 mM tris, pH 8.5 | 0.5 M | 5% DTT | 23.7 |
| 50 mM tris, pH 8.5 | 0.25 M | 5% DTT | 32.5 |

### Example 45

### Binding of Linearized pUC18 DNA with Tributylphosphonium Beads of Example 1 and Release with Different Elution Compositions Containing Na, K or Mg Ions

A 10 mg sample of beads was rinsed with 200 μL of water. A solution of 2 μg of linearized pUC18 DNA in 200 μL of water was added to 10 mg of the beads and the mixture gently shaken for 25 min. The mixture was spun down and the supernatant collected. The beads were rinsed with 2×200 μL of water and the water discarded. DNA was eluted by incubating the beads with 200 μL of various reagent compositions described in the table below at room temperature for 25 min. The mixture was spun down and the eluent removed for analysis by fluorescence and gel electrophoresis.

| Buffer | [Salt] | Org. Solvent | % Eluted |
|---|---|---|---|
| 50 mM tris, pH 8.5 | 1.25 M NaCl | 5% DTT | 53.6 |
| 50 mM tris, pH 8.5 | 1.25 M KCl | 5% DTT | 60.0 |
| 50 mM tris, pH 8.5 | 1.25 M MgCl₂ | 5% DTT | 11.5 |
| 50 mM tris, pH 8.5 | 0.75 M NaCl | 5% DTT | 67.8 |
| 50 mM tris, pH 8.5 | 0.75 M KCl | 5% DTT | 64.4 |
| 50 mM tris, pH 8.5 | 0.75 M MgCl₂ | 5% DTT | 17.6 |
| 50 mM tris, pH 8.5 | 0.1 M MgCl₂ | 5% DTT | 25.6 |
| 50 mM tris, pH 8.5 | none | 5% DTT | N.D. |
| 50 mM tris, pH 8.5 | none | none | N.D. |

(N.D.—not detected)

### Example 46

### Binding of Linearized pUC18 DNA with Tributylphosphonium Beads of Example 1 and Release with Different Elution Compositions Containing Various Ions

A 10 mg sample of beads was rinsed with 200 μL of water. A solution of 2 μg of linearized pUC18 DNA in 200 μL of water was added to 10 mg of the beads and the mixture gently shaken for 25 min. The mixture was spun down and the supernatant collected. The beads were rinsed with 2×200 μL of water and the water discarded. DNA was eluted by incubating the beads with 200 μL of various reagent compositions described in the table below at room temperature for 30 min. The mixture was spun down and the eluent removed for analysis by gel electrophoresis.

| Buffer | [Salt] | Org. Solvent | % Eluted |
|---|---|---|---|
| 50 mM tris, pH 8.5 | 0.75 M LiCl | 5% DTT | 77.0 |
| 50 mM tris, pH 8.5 | 0.75 M CaCl₂ | 5% DTT | 76.3 |
| 50 mM tris, pH 8.5 | 0.75 M CsCl | 5% DTT | 73.9 |
| 50 mM tris, pH 8.5 | 0.75 M ZnCl₂ | 5% DTT | 47.2 |
| 50 mM tris, pH 8.5 | 0.75 M NH₄Cl | 5% DTT | 49.6 |
| 50 mM tris, pH 8.5 | 0.1 M LiCl | 5% DTT | N.D. |
| 50 mM tris, pH 8.5 | 0.1 M CaCl₂ | 5% DTT | 62.3 |
| 50 mM tris, pH 8.5 | 0.1 M CsCl | 5% DTT | N.D. |
| 50 mM tris, pH 8.5 | 0.1 M ZnCl₂ | 5% DTT | N.D. |
| 50 mM tris, pH 8.5 | 0.1 M NH₄Cl | 5% DTT | N.D. |

### Example 47

### Release of Bound pUC18 DNA from Cleavable Magnetic Tributylphosphonium Beads of Example 52 with Buffer Composition Used Directly in PCR

A 10 mg sample of the magnetic phosphonium beads of example 52 was rinsed with 400 μL of THF followed by 2×100 μL of water. A solution of 2 μg of uncut pUC18 DNA in 200 μL of lysate was added to 10 mg of beads and the mixture gently shaken for 5 min. Lysate buffer comprised a 1:1:1 mixture of three buffers; S1: 50 mM tris, pH 8.0, 10 mM EDTA; S2: 0.2 M NaOH solution containing 1% SDS; S3: 0.3 M KOAc, 0.2 M HCl. The mixture was magnetically separated and the supernatant collected. The beads were rinsed with 2×200 μL of water and the water discarded. DNA was eluted by incubating the beads with 200 μL of a concentrated buffer containing 400 mM tris-HCl, pH 8.4, 1 M KCl and 50 mM MgCl₂ at 37° C. for 5 min. Fluorescence assay revealed that 100% of the plasmid DNA was bound to the beads; 41% was eluted.

The eluent containing DNA was diluted in various ratios 1:10 and 1:20 ratios with water and PCR amplified using 30 cycles of 94° C.-1 min, 60° C.-1 min, 72° C.-1 min. The 1:10 and 1:20 dilutions successfully amplified by PCR.

### Example 48

### Isolation of Plasmid DNA from Bacterial Culture with Polymer Beads of Example 1 Using Various Buffer Compositions

An E. coli culture was grown overnight. A 20 mL portion was centrifuged at 6000×g for 15 min at 4° C. to pellet the cells. The pellet was resuspended in 4 mL of 50 mM tris, pH 8.0, 10 mM EDTA, containing 100 μg/mL RNase A. Then 4 mL of 0.2 M NaOH solution containing 1% SDS was added to the mixture which was gently mixed and kept for 4 min at room temperature. Next, 4 mL of 0.3 M KOAc, containing 0.2 M HCl, cooled to 4° C., was added, the solution mixed and allowed to stand for 10 min to precipitate SDS. The precipitate was filtered off and the filtrate was collected.

Lysate (200 μL) was mixed with 10 mg of the beads of example 1 and incubated for 5 min. After binding, the beads were spun down and the supernatants removed. The bead samples were washed with 2×200 μL of water and then eluted with compositions as detailed in the table.

| Buffer | Org. Solvent | Yield (μg) |
|---|---|---|
| 1.25 M tris, pH 8.5 | 15% 2-propanol | 3.5 |
| 1.25 M tris, pH 8.5 | 5% DTT | 2.1 |
| 0.05 M tris, pH 8.5, | 5% DTT | 1.5 |
| (+0.1 M MgCl₂) | | |

### Example 49

### Synthesis of a Polystyrene Polymer Containing Dimethylphosphonium Groups

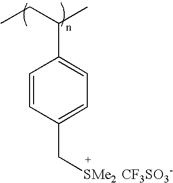

Merrifield peptide resin (Sigma, 1.1 meq/g, 2.0 g) was and sodium thiomethylate (2.24 g, 10 equivalents) were added to a 250 mL flask along with 60 mL of anh. DMF. The mixture was placed under Ar and stirred at room temperature for 15 days. The slurry was filtered and the resulting solids were washed with 50 mL of DMF, 200 mL of water, 200 mL of methanol, and 200 mL of CH₂Cl₂. The resin was air-dried (2.12 g).

The thiomethylated polymer (0.637 g) in 100 mL of CH₂Cl₂ was put under Ar and reacted with 1.6 mL of methyl triflate. After stirring for 13 days, the mixture was filtered and the solid washed with 200 mL of CH₂Cl₂, 200 mL of methanol and 200 mL of CH₂Cl₂. Air drying left 2.09 g of white solid.

### Example 50

### Release of pUC18 DNA Bound onto Polymer Beads of Example 49 Using Various Buffer Compositions

A 10 mg sample of the sulfonium beads of example 49 was rinsed with 300 μL of THF followed by 2×100 μL of water. A solution of 2 μg of linearized pUC18 DNA in 200 μL of water was added to 10 mg of beads and the mixture gently shaken for 15 min. The beads were spun down and the supernatant discarded. The beads were rinsed with 2×200 μL of water and the water discarded. DNA was eluted by incubating the beads with 200 μL of the buffer below at room temperature for 15 min. Fluorescence assay revealed that 100% of the plasmid DNA was bound to the beads; 56% was eluted.

| Buffer | Org. Solvent | Salt | % Eluted |
|---|---|---|---|
| 50 mM tris. pH 8.5, | 5% DTT | 0.75 M NaCl | 56 |

### Example 51

### Synthesis of 4′-Hydroxyphenyl 4-chloromethylthiobenzoate

A 3 L flask was charged with 100.9 g of 4-chloromethylbenzoic acid and 1.2 L of thionyl chloride. the reaction was refluxed for 4 h, after which the thionyl chloride was removed under reduced pressure. Residual thionyl chloride was removed by addition of CH₂Cl₂ and evaporation under reduced pressure.

A 3 L flask containing 113.1 g of 4-chloromethylbenzoic acid chloride was charged with 98.17 g of 4-hydroxythiophenol and 1.5 L of CH₂Cl₂. Argon was purged in and 67.75 mL of pyridine added. After stirring over night, the reaction mixture diluted with 1 L of CH₂Cl₂ and extracted with 5 L of water. The water layer was back extracted with CH₂Cl₂. The combined CH₂Cl₂ solutions were dried over sodium sulfate and concentrated to a solid. The solid was washed with 500 mL of CH₂Cl₂, filtered and air dried. ¹H NMR (acetone-d₆): δ 4.809 (s, 2H), 6.946-6.968 (d, 2H), 7.323-7.346 (d, 2H), 7.643-7.664 (d, 2H), 8.004-8.025 (d, 2H).

### Example 52

### Synthesis of Magnetic Silica Particles Functionalized with Polymethacrylate Linker and Containing Tributylphosphonium Groups and Cleavable Arylthioester Linkage

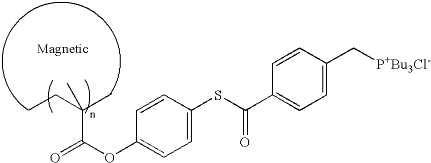

Magnetic carboxylic acid-functionalized silica particles (Chemicell, SiMAG-TCL, 1.0 meq/g, 1.5 g) were placed in 20 mL of thionyl chloride and refluxed for 4 hours. The excess thionyl chloride was removed under reduced pressure. The resin was resuspended in 25 mL of CHCl₃ and the suspension dispersed by ultrasound. The solvent was evaporated and ultrasonic wash treatment repeated. The particles were dried under vacuum for further use. The acid chloride functionalized particles were suspended in 38 mL of CH₂Cl₂ along with 388 mg of diisopropylethylamine. 4′-Hydroxyphenyl 4-chloromethylthiobenzoate (524 mg) was added and the sealed reaction flask left on the shaker over night. The particles were transferred to a 50 mL plastic tube and washed repeatedly, with magnetic separation, with portions of CH₂Cl₂, CH₃OH, 1:1 CH₂Cl₂/CH₃OH, and then CH₂Cl₂. Wash solutions were monitored by TLC for removal of unreacted soluble starting materials. The solid was air dried before further use.

The resin (1.233 g) was suspended in 20 mL of CH₂Cl₂ under argon. Tributylphosphine (395 mg) was added and the slurry shaken for 7 days. The particles were transferred to a 50 mL plastic tube and washed 4 times with 40 mL of CH₂Cl₂ followed with 4 washes of 40 mL of MeOH and 4 times with 40 mL of CH₂Cl₂. The resin was then air dried yielding 1.17 g of a light brown solid.

### Example 53

### Synthesis of Polymethacrylate Polymer Particles Containing Tributylphosphonium Groups and Cleavable Arylthioester Linkage

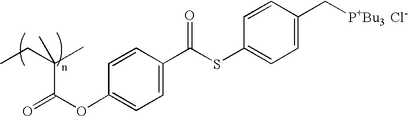

Poly(methacryloyl chloride) particles (1.0 meq/g, 1.5 g) were placed in 75 mL of CH₂Cl₂ containing 2.45 g of diisopropylethylamine. Triethylamine (1.2 g) was added. 4′-Hydroxyphenyl 4-chloromethylthiobenzoate (4.5 g) was added and the sealed reaction mixture was stirred overnight at room temperature. The slurry was filtered and the resin washed with 10 mL of CH₂Cl₂, 200 mL of acetone, 200 mL of MeOH, 2×100 mL of 1:1 THF/CH₂Cl₂, 250 mL of THF, 250 mL of CH₂Cl₂, 250 mL of hexane. The resin was air dried for further use.

The resin (1.525 g) was suspended in 25 mL of CH₂Cl₂ under argon. Tributylphosphine (1.7 g) was added and the slurry stirred for 4 days. The resin was filtered and washed 4 times with 225 mL of CH₂Cl₂ followed by 175 mL of hexane. The resin was then air dried yielding 1.68 g of solid.

In a similar manner, polymer particles containing trimethylphosphonium groups were also prepared.

### Example 54

### Release of Bound pUC18 DNA from Cleavable Tributylphosphonium Beads of Example 53 with Buffer Composition

Plasmid DNA was bound on the particles of example 56 according to the protocol described in example 49. DNA was eluted by incubating the particles in 100 μL of 1.25 M tris, pH 8.5 buffer containing 5% DTT at 37° C. for 5 min.

Fluorescence assay showed that 100% of the plasmid DNA was bound to the beads; 55% was eluted.

1. A method of isolating from a sample a nucleic acid selected from the group consisting of oligonucleotides, DNA, RNA or a synthetic DNA analog comprising:
a) providing a solid phase binding material;
b) combining the solid phase with the sample containing the nucleic acid to bind the nucleic acid to the solid phase binding material;
c) separating the sample from the solid phase binding material; and
d) releasing the nucleic acid from the solid phase by elution with a composition comprising an aqueous amine buffer solution having a pH of 7-9 wherein the concentration of the amine is at least 0.01 M, 0.1-3 M of a monovalent or divalent halide salt or acetate salt, and 0.01-50% of a hydrophilic organic co-solvent selected from the group consisting of ethylene glycol, propylene glycol, glycerol, water soluble mercaptans, 2-mercaptoethanol, dithiothreitol, furfuryl alcohol, 2,2,2-trifluoroethanol, acetone, THF, and p-dioxane.

2. The method of claim 1 wherein the solid phase is selected from the group consisting of silica, glass, insoluble synthetic polymers, and insoluble polysaccharides.

3. The method of claim 1 wherein the solid phase has a nucleic acid binding portion comprising a quaternary phosphonium group PR₃⁺X⁻ wherein R is selected from the group consisting of C₁-C₂₀ alkyl, aralkyl and aryl groups, and wherein X is an anion.

4. The method of claim 1 wherein the solid phase has a nucleic acid binding portion comprising a ternary sulfonium group of the formula SR₂⁺X⁻ where R is selected from the group consisting of C₁-C₂₀ alkyl, aralkyl and aryl groups, and wherein X is an anion.

5. The method of claim 1 wherein the solid phase has a nucleic acid binding portion comprising a quaternary ammonium group NR₃⁺X⁻ wherein R is selected from the group consisting of C₁-C₂₀ alkyl, aralkyl and aryl groups, and wherein X is an anion.

6. The method of claim 1 wherein the solid phase material further comprises a magnetic core portion.

7. The method of claim 1 wherein the salt is selected from halides and acetate salts of NH₄, Li, Na, K, Rb, Cs, Ca, Mg, and Zn.

8. The method of claim 7 wherein the salt is present at a concentration of at least 0.1 M.

9. The method of claim 1 wherein the hydrophilic organic co-solvent is selected from 2-mercaptoethanol and dithiothreitol.

10. The method of claim 1 wherein the amine is selected from the group consisting of aliphatic amines, aliphatic amino acids, aliphatic amino alcohols and sulfonated aliphatic amines.

11. The method of claim 1 wherein the nucleic acid is human genomic DNA and the sample is a bodily fluid.

12. The method of claim 1 wherein the nucleic acid is plasmid DNA and the sample is a cell culture.

13. The method of claim 1 wherein the solid phase further comprises a cleavable linker portion that links the solid support portion to the nucleic acid binding portion.

14. A method of isolating from a sample a nucleic acid selected from the group consisting of oligonucleotides, DNA, RNA or a synthetic DNA analog comprising:
a) providing a solid phase binding material;
b) combining the solid phase with the sample containing the nucleic acid to bind the nucleic acid to the solid phase binding material;
c) separating the sample from the solid phase binding material; and
d) releasing the nucleic acid from the solid phase by elution with a composition comprising an aqueous amine buffer solution having a pH of 7-9 wherein the concentration of the amine is at least 0.1 M, and 0-50% of a hydrophilic organic co-solvent selected from the group consisting of C₁-C₄ alcohols, ethylene glycol, propylene glycol, glycerol, water soluble mercaptans, 2-mercaptoethanol, dithiothreitol, furfuryl alcohol 2,2,2-trifluoroethanol, acetone, THF, and p-dioxane.

15. The method of claim 14 wherein the concentration of the buffer is at least 0.4 M.

16. The method of claim 14 wherein the concentration of the buffer is at least 1 M.

17. The method of claim 14 wherein the amine is selected from the group consisting of aliphatic amines, aliphatic amino acids, aliphatic amino alcohols and sulfonated aliphatic amines.

18. The method of claim 14 wherein the hydrophilic organic co-solvent is selected from 2-mercaptoethanol and dithiothreitol.

19. The method of claim 14 wherein the solid phase has a nucleic acid binding portion comprising a quaternary onium group selected from the group consisting of a quaternary phosphonium group PR₃⁺X⁻ wherein R is selected from the group consisting of C₁-C₂₀ alkyl, aralkyl and aryl groups, a ternary sulfonium group of the formula SR₂⁺X⁻ where R is selected from the group consisting of C₁-C₂₀ alkyl, aralkyl and aryl groups, and a quaternary ammonium group NR₃⁺X⁻ wherein R is selected from the group consisting of C₁-C₂₀ alkyl, aralkyl and aryl groups, and wherein X is an anion.

20. The method of claim 14 wherein the solid phase material further comprises a magnetic core portion.

21. The method of claim 14 wherein the nucleic acid is human genomic DNA and the sample is a bodily fluid.

22. The method of claim 14 wherein the nucleic acid is plasmid DNA and the sample is a cell culture.

23. A method of isolating from a sample a nucleic acid selected from the group consisting of oligonucleotides, DNA, RNA or a synthetic DNA analog comprising:
a) providing a solid phase binding material which has a nucleic acid binding portion comprising either a quaternary phosphonium group PR₃⁺X⁻ wherein R is selected from the group consisting of C₁-C₂₀ alkyl, aralkyl and aryl groups, and wherein X is an anion, or a ternary sulfonium group of the formula SR₂⁺X⁻ where R is selected from the group consisting of C₁-C₂₀ alkyl, aralkyl and aryl groups, and wherein X is an anion;
b) combining the solid phase with the sample containing the nucleic acid to bind the nucleic acid to the solid phase binding material;
c) separating the sample from the solid phase; and
d) releasing the nucleic acid from the solid phase by elution with a composition comprising an aqueous amine buffer solution having a pH of 7-9 wherein the concentration of the amine is at least 0.01 M, 0.1-3 M of a monovalent or divalent halide salt or acetate salt, and 0.01-50% of a hydrophilic organic co-solvent selected from the group consisting of C₁-C₄ alcohols, ethylene glycol, propylene glycol, glycerol, water soluble mercaptans, 2-mercaptoethanol, dithiothreitol, furfuryl alcohol 2,2,2-trifluoroethanol, acetone, THF, and p-dioxane.

24. The method of claim 23 wherein the solid phase material further comprises a magnetic core portion.

25. The method of claim 23 wherein the amine is selected from the group consisting of aliphatic amines, aliphatic amino acids, aliphatic amino alcohols and sulfonated aliphatic amines.

26. The method of claim 23 wherein the salt is selected from halides and acetate salts of NH₄, Li, Na, K, Rb, Cs, Ca, Mg, and Zn.

27. The method of claim 23 wherein the hydrophilic organic co-solvent is selected from 2-mercaptoethanol and dithiothreitol.

28. The method of claim 27 wherein the concentration of the hydrophilic organic co-solvent is at least 1%.

29. The method of claim 23 wherein the nucleic acid is human genomic DNA and the sample is a bodily fluid.

30. The method of claim 23 wherein the nucleic acid is plasmid DNA and the sample is a cell culture.

31. The method of claim 23 wherein the solid phase further comprises a cleavable linker portion that links the solid support portion to the nucleic acid binding portion.
